(19) United States
(12) Patent Application Publication
(10) Pub. No.: US 2011/0143385 A1
(43) Pub. Date: Jun. 16, 2011

(54) PLASMA MEMBRANE VESICLES AND METHODS OF MAKING AND USING SAME

(75) Inventors: Brigitte Bauer, Goteborg (SE); Max Davidson, Goteborg (SE); Owe Orwar, Hovas (SE); Anders Karlsson, Molnlycke (SE); Roger Karlsson, Molndal (SE)

(73) Assignee: NANOXIS AB, Goteborg (SE)

(21) Appl. No.: 12/909,375

(22) Filed: Oct. 21, 2010

Related U.S. Application Data
(63) Continuation of application No. PCT/IB2009/051622, filed on Apr. 21, 2009.
(60) Provisional application No. 61/046,479, filed on Apr. 21, 2008.

(51) Int. Cl. C12Q 1/37; C12N 5/071; C12N 13/00; C12Q 1/02; C12Q 1/34

(52) U.S. Cl. 435/23; 435/375; 435/173.1; 435/29; 435/18

(57) ABSTRACT

The instant invention provides plasma membrane vesicles, methods of making the same, and method of using the plasma membrane vesicles.

### RELATED APPLICATIONS

This application claims the benefit of U.S. Provisional Application No. 61/046,479, filed Apr. 21, 2008, the entire contents for which are expressly incorporated herein by reference.

### BACKGROUND OF THE INVENTION

Biomembranes or biological membranes are the walls that separate the cell from its surrounding environment (i.e. the plasma membrane) and also construct internal structures inside the cell, such as organelles (Golgi complex, endoplasmic reticulum and mitochondria, for example) and the nucleus. The functions of this wall structure include ways for the cell to regulate and control the influx and efflux of material, package and transport material inside the cell between different organelles, provide specific transport highways for certain reagents or signaling substances and of course to provide containment through formation of compartments inside the cellular volume (Lehninger et al., 1993).

Efficient proteomic and lipidomic analysis of plasma membranes is enormously important in order to elucidate its function and to find new targets for drug development, as plasma membrane proteins account for ˜70% of all known drug targets. (e.g. ion channels, and G protein-coupled receptors). Methods to analyze membrane proteomes are constantly under development and new protocols emerge on a regular basis and a current trend is an increasing interest in the lipid constituents of membranes and their function.

Difficulties in membrane proteome and lipidome analysis arise primarily due to the distribution of the lipids and membrane proteins in subcellular compartments as well as in the plasma membrane. In order to assign the identified proteins and lipids to their original location, the membrane sub locations have to be distinguishable. To accomplish that, it is usually necessary to separate subcellular organelles, typically done by cell lysis followed by differential- and/or density gradient centrifugation. This method relies on separation by the inherent density of the organelles, determined primarily by the lipid:protein ratio and composition. A certain degree of enrichment of organelles can be achieved, but there is often compositional overlap between fractions since membranes of different organelles can have very similar densities. Thus, using such protocols, the laws of physics refute a complete separation of different membrane protein sources. Another complication is the fact that the endomembrane system is interconnected. Vesicle, and tubule traffic shuttles materials through the secretory and endocytic pathways, again leading to overlaps in the membrane protein and lipid distribution. Many membrane proteins are therefore assigned to multiple cellular locations, as has been observed e.g. by protein correlation profiling. Various methods have been developed to isolate plasma membranes for proteomic studies, e.g. affinity enrichment, but these methods share in common the problem of contamination, where other organelles still account for ˜30-40% of the identified membrane proteins, hampering identification of unique plasma membrane proteins. The same naturally applies to the analysis of the plasma membrane lipids. If one examines the biomembrane composition between different cell types or even organelles within the same cell type, the variability and number of different lipid species is striking (Schmidt and MacKinnon, 2008). The diversity of lipid species in biomembranes is coupled to the function of the membrane to some extent, for example, some proteins are only functional in the presence of certain lipids. Also, many processes involve electrostatic control of protein adsorption, through charged lipids in the biomembrane, which itself can mediate a reaction taking place on the surface of the biomembrane. Taking the examination of the biomembrane even further, one can also deduce an asymmetry of the distribution of the various lipid species between the two monolayers of the plasma membrane. For example, while phosphatidyl choline is mainly found in the outer monolayer of the plasma membrane, the majority of both phosphatidyl ethanolamine and phosphatidyl serine are situated in the inner monolayer (Langner and Kubica, 1999). This most probably reflects the duality of the monolayers function, the outer monolayer providing more or less an inert barrier for the surrounding environment, while the inner surface provides sites for reactions to occur by the net charge that arises from, for example, the phosphatidyl serine component.

Accordingly, a need exists to find methods and compositions to facilitate the study and characterization of membrane proteins and lipids in the plasma membrane.

### SUMMARY OF THE INVENTION

The instant invention is based, at least in part, on the inventors' discoveries of methods of making high purity plasma membrane vesicles and methods of using the same. In one aspect, the invention provides, methods for producing plasma membrane vesicles comprising, contacting a cell with a vesiculation agent, thereby producing plasma membrane vesicles. In one embodiment, the methods further comprise mechanically agitating the cells.

In one embodiment, the cells are adherent cells. In another embodiment, the cells are in suspension. In a related embodiment, the cells are mammalian cells, e.g., human cells.

In one embodiment, the vesiculation agent comprises a sulfhydryl blocking agent, e.g., is formaldehyde, pyruvic aldehyde, acetaldehyde, glyoxal, glutaraldehyde, acrolein, methacrolein, pyridoxal, N-ethyl malemide (NEM), malemide, chloromercuribenzoate, iodoacetate, potassium arsenite, sodium selenite, thimerosal (merthiolate), benzoyl peroxide, cadmium chloride, hydrogen peroxide, iodosobenzoic acid, meralluride sodium, (mercuhydrin), mercuric chloride, mercurous chloride, chlormerodrin (neohydrin), phenylhydrazine,potassium tellurite, sodium malonate, p-arsenosobenzoic acid, 5,5′-diamino-2,2′-dimethyl arsenobenzene, N,N′-dimethylene sulfonate disodium salt, iodoacetamide, oxophenarsine (mapharsen), auric chloride, p-chloromercuribenzoic acid, p-chloromercuriphenylsulfonic acid, cupric chloride, iodine merbromin (mercurochrome)porphyrindine, potassium permanganate, mersalyl (salyrgan), silver nitrate, strong silver protein (protargol), and uranyl acetate.

In a specific embodiment, the vesiculation agent comprises dithiothreitol (DTT) and formaldehyde.

In alternative embodiments, the vesiculation agent is a cell toxin, e.g., cytochalasin B or melittin.

In other embodiments, the cells are mechanically agitated by a shaker, or by ultrasonication.

In other embodiments, the methods further comprise washing cells to remove culture medium prior to contacting cells with the vesiculation agent.

In other embodiments, the methods further comprise purifying the plasma membrane vesicles, e.g., by any one or more of filtering, density gradient centrifugation, or dialysis.

In other embodiments, the methods of the invention further provide methods of making high purity plasma membrane vesicles comprising one or more of the following steps:
contacting the plasma membrane vesicles with an alkylating and reducing agent;
contacting the plasma membrane vesicles with an alkaline solution;
using ultrasonication on the plasma membrane vesicles to release intravesicular contaminants;
using ultracentrifugation on the plasma membrane vesicles to clean the plasma membrane vesicles; and
washing the plasma membrane vesicles with a buffer solution.

In related embodiments, the alkylation reagent is iodoacetamide. In further related embodiments, the alkaline solution is at least pH 11. In yet further embodiments, the alkaline solution is Na₂CO₃ or NaOH.

In other embodiments, the diameter of the plasma membrane vesicles is 20 μm or less, or 10 μm or less.

In some embodiments, the plasma membrane vesicles comprise transmembrane proteins, e.g., transmembrane alpha-helix proteins, transmembrane beta-barrel proteins, lipid anchored membrane proteins, and peripheral membrane proteins.

In exemplary embodiments, the transmembrane proteins are selected from the group consisting of enzymes, transporters, receptors, channels, cell adhesion proteins, G proteins, GTPases

In other embodiments, the plasma membrane vesicles comprise lipid anchored proteins.

In other embodiments, the plasma membrane vesicles comprise lipids of specific composition related to the cell type of the origin of the plasma membrane vesicles.

In another aspect, the invention provides methods for analyzing the membrane proteome of a cell by contacting the plasma membrane vesicle of described herein with one or several proteases, or several proteases in series, analyzing the peptides generated by the protease, thereby analyzing the membrane proteome of a cell.

In specific embodiments, the protease is a serine protease, e.g., trypsin or chymotrypsin.

In additional embodiments, the methods further comprise isolating the protein fragments. In exemplary embodiments, the peptide fragments are analyzed by mass spectrometry.

In another aspect, the invention provides methods of identifying a modulator of a transmembrane protein by transforming a cell with a nucleic acid molecule encoding a protein of interest, producing plasma membrane vesicles by the methods described herein, contacting the plasma membrane vesicles with a candidate modulator, determining if the candidate modulator is capable of modulating the transmembrane protein, thereby identifying a modulator of a transmembrane protein. In a related embodiment, the ability of the candidate modulator to modulate the transmembrane protein is determined by measuring the activity of a reporter gene.

In another aspect, the invention provides methods of determining the effect of a compound on the transmembrane proteome by contacting a cell with a compound; producing plasma membrane vesicles by the methods described herein, analyzing the polypeptides present in the plasma membrane vesicles, thereby determining the effect of a compound on the transmembrane proteome. In exemplary embodiments the compound is a small molecule, polypeptide, peptide, nucleic acid molecule, RNAi, shRNA, or miRNA.

In related embodiments, the methods further comprise contacting the plasma membrane vesicle with a protease. In another embodiment, the methods further comprise analyzing the peptides produced by the protease by mass spectrometry.

In another aspect, the invention provides methods of analyzing the proteins in plasma membrane vesicles described herein by affixing the plasma membrane vesicles to a surface, contacting the plasma membrane vesicles with one or more proteases, and analyzing the peptides generated to determine the identity of the proteins.

In one embodiment, the surface is in a microfluidic device. In another embodiment, the peptides are analyzed by mass spectroscopy.

In another aspect, the invention provides methods for analyzing the lipid components of the plasma membrane by contacting a cell with a compound; producing plasma membrane vesicles by the methods described herein, and extracting the lipid components for further analysis.

In a related aspect, the extraction of lipid constituents can be performed by a plethora of methods depending on the lipid target constituents.

In another aspect, the invention provides methods of determining the effect of the lipid composition when reconstituting transmembrane proteins in the extracted plasma membrane lipids. In this aspect, cells are contacted by a compound, producing plasma membrane vesicles by the methods described herein, the lipid components are extracted also by methods described herein and finally the membrane proteins are reconstituted in the extracted lipid. In a related aspect, reconstitution refers to the extraction of membrane proteins from their natural membrane with the use of e.g. detergents and inserting them into a lipid membrane environment.

In another embodiment, the invention provides methods and applications for studying transport across plasma membranes, uptake studies and membrane interaction studies of substances with the plasma membrane. In a related aspect, the substances can be, but not limited to, peptides, proteins, sugars, cholesterol and various forms of DNA and RNA.

In another aspect, the invention provides populations of monodisperse plasma membrane vesicles. In exemplary embodiments, the plasma membrane vesicles are from 5 μm to 25 nm in diameter, from 50 μm to 500 μm in diameter, or from 100 μm to 200 μm in diameter.

In a related embodiment, the population has been enriched for a given membrane protein, e.g., a transmembrane protein, or a lipid anchored proteins. In related embodiment, the population is enriched by immunohistochemistry or affinity purification.

In one embodiment, the plasma membrane vesicles are free from organelles or cytoskeletal structures.

### DESCRIPTION OF THE DRAWINGS

FIG. 1 After growing a cell culture to confluence (A) the growth medium is removed by aspiration and washed 2 times (B) to remove residual growth medium contaminants. (C) Vesiculation solution is added to the cell layer (D) plasma membrane vesicles are formed at the cell surface and bud off into the solution during incubation. The flasks are agitated to promote PMV shedding (E) The plasma membrane vesicle solution is carefully aspirated from the cell layer and transferred into a conical tube (F) to obtain a crude plasma membrane vesicle solution.

FIG. 2 (A-B) The harvested cell-containing plasma membrane vesicle solution is underlaid with a 2M sucrose solution to provide a high density phase. (C) The solution is centrifuged at low-speed using a swing-out rotor. During centrifugation only detached cells and cell debris are pelleted in the high-density sucrose phase, whereas plasma membrane vesicles remain in the low density buffer phase. (D) The upper phase containing plasma membrane vesicles is carefully aspirated, transferred into a large cutoff dialysis membrane and placed in HEPES buffer for dialysis. (F) During dialysis, vesiculation agents and low MW proteins are removed, yielding an ultrapure plasma membrane vesicle solution.

FIG. 3 (A-B) The purified plasma membrane vesicles are exposed to reducing and alkylating agents to expose cleavage sites and prevent protein aggregation. (C) The plasma membrane vesicle solution is washed with Na₂CO₃ at high pH to disrupt non-covalent protein-protein interactions, dissociating cytosolic proteins from the membrane. Sonication disrupts plasma membrane vesicles and allows release of their cytosolic contents. (D) the plasma membrane vesicles are ultracentrifuged, and the supernatant is aspirated (E-F) to remove contaminants released from the PMV interior. (G) The membrane pellet is rinsed and sonicated in buffer to obtain an ultrapure small-sized plasma membrane vesicle solution.

FIG. 4 (A-B) Processed plasma membrane vesicles are immobilized on the flowcell surface by injecting the plasma membrane vesicle solution via the inlet nozzle. (C-D) After injection of protease, surface-exposed domains of membrane proteins are cleaved yielding a defined set of peptides. These are eluted (D) from the chip via the outlet nozzle. (E) The eluted peptide sample is processed and analyzed via LC-MS/MS.

FIG. 5. Comparison of identified membrane proteins in purified plasma membrane vesicles and microsomes. In plasma membrane vesicle, 32 out of 43 membrane proteins are associated uniquely to the plasma membrane, compared to only 17 out of 79 in microsomes. Overall, ˜44% of microsomal membrane proteins are plasma membrane-associated, whereas PMV analysis resulted in 93% PM-associated proteins.

FIG. 6. Classes of plasma membrane proteins found in plasma membrane vesicles. GTPases comprise the largest fraction, followed by G-proteins and proteins related to cell adhesion functionality. Knowledge of the membrane protein setup of plasma membrane vesicles holds promise for the development of activity assays in single plasma membrane vesicles.

FIG. 7. Comparison of subcellular distribution for anchored membrane proteins with unique location. (A) distribution in plasma membrane vesicle membranes (B) distribution in microsome membranes

FIG. 8. The structure of glycerophospholipids. The backbone of these structures is a glycerol molecule that is linked to two alkyl chains or fatty acids of various degree of saturation through ester bonds. The third link through the phosphate molecule defines the head-group of the lipids. The figure also states the net charge of the lipid at pH 7. Phosphatidyl choline is a zwitterionic lipid, which means that the lipid contains both negative and positive charges that cancel each other at neutral pH values.

FIG. 9. The structure of sphingolipids, containing a long-chain amine alcohol sphingosine as a backbone, together with a long-chain fatty acid and a polar head alcohol, which can be further linked to other polar head-groups via, for example, a phosphodiester linkage. The simplest compound in this group is the ceramide and the image also shows examples from the three different groups of the sphingolipids: sphingomyelins, glycolipids and gangliosides. The symbols for sugars used in this image are: Glc, D-glucose; Gal, D-galactose; GalNAc, N-acetyl-D-galactosamine; NeuNAc, N-acetylneuraminic acid (sialic acid).

### DETAILED DESCRIPTION OF THE INVENTION

The instant invention provides plasma membrane vesicles and methods of making plasma membrane vesicles. The methods provided herein allow one of skill in the art to make plasma membrane vesicles from any cell type they choose. In certain embodiments, the plasma membrane vesicles described herein can be used with the device as described in WO 2006/068619, the contents of which are expressly incorporated herein by reference.

Preparation of Plasma Membrane Vesicles

The instant invention provides for the production and use plasma membrane vesicles comprising membrane proteins. In one embodiment, the plasma membrane vesicles are free of organelles or cell matrix material.

In one aspect, the instant methods allow for production of high purity and/or monodisperse plasma membrane vesicles.

By “monodisperse” it is meant a population of plasma membrane vesicles that are of similar size. In preferred embodiments, the diameter of the members of a population of plasma membrane vesicles of the invention are within about 20%, 15%, 10%, 5%, 4%, 3%, or 2% of each other.

Plasma membrane vesicles are also known as blebs. Blebs are little bud-like protrusions formed in the cell wall, outer membrane, cytoplasmic, and/or plasma membrane of a cell. When cultured under selected conditions described hererin the membrane vesicles break away from the whole cell into the medium. The membrane vesicles are generally spherical, possess a bilayer, and have a diameter of about 1 μm to about 100 μM.

The instant methods rely on contacting a cell with an agent that induces vesculation. In certain embodiments, the vesiculation agent comprises a sulfhydryl blocking agent, e.g., formaldehyde, pyruvic aldehyde, acetaldehyde, glyoxal, glutaraldehyde, acrolein, methacrolein, pyridoxal, N-ethyl malemide (NEM), malemide, chloromercuribenzoate, iodoacetate, potassium arsenite, sodium selenite, thimerosal (merthiolate), benzoyl peroxide, cadmium chloride, hydrogen peroxide, iodosobenzoic acid, meralluride sodium, (mercuhydrin), mercuric chloride, mercurous chloride,chlormerodrin (neohydrin), phenylhydrazine, potassium tellurite, sodium malonate, p-arsenosobenzoic acid, 5,5′-diamino-2,2′-dimethyl arsenobenzene, N,N′-dimethylene sulfonate disodium salt, iodoacetamide, oxophenarsine (mapharsen), auric chloride, p-chloromercuribenzoic acid, p-chloromercuriphenylsulfonic acid, cupric chloride, iodine merbromin (mercurochrome)porphyrindine, potassium permanganate, mersalyl (salyrgan), silver nitrate, strong silver protein (protargol), or uranyl acetate. In other embodiments the vesculation agent is a combination of these agents, e.g., dithiothreitol (DTT) and formaldehyde acting in concert.

In other embodiments, the vesiculation agent is a cell toxin, e.g., cytochalasin B or melittin.

In some embodiments of the invention, the cells are mechanically agitated in order to increase the amount of vesicle formation. The mechanical agitation can be, for example, a shaker or ultrasonication.

Once the plasma membrane vesicles are formed, they can be purified if so desired. There are many ways to purify the plasma membrane vesicles including, but not limited to, filtering, density gradient centrifugation, or dialysis. In some instances combination of several of these methods is desirable.

In order to produce ultrapure plasma membrane vesicles, one or more of the following purification and manipulation steps may be performed: alkylation and reduction of membrane proteins, alkaline wash to disrupt non-covalent protein-protein interactions, ultrasonication to release intravesicular contaminants and form small vesicles, ultracentrifugation to clean plasma membrane vesicle fraction, rinsing and dispersion in ammonium bicarbonate buffer.

Reduction of membrane proteins can be accomplished by contacting them with for example, dithiothritol, tris(carboxyethyl)phosphine (TCEP) or tributylphosphine (TBP) to replace DTT, or a combination of iodoethanol and triethylphosphineDTT and alkylation can be preformed with iodoacetamide to break disulfide bonds. This allows for more cleavage sites available for digestion and reduces protein aggregation.

The plasma membrane vesicles can be washed with a high pH solution or high salt solution to disrupt non-covalent protein-protein interactions. This step will also dissociate cytosolic proteins from the membrane.

The plasma membrane vesicles can be exposed to ultrasonic waves to release intravesicular contaminants and form smaller vesicles. This purification step includes extensive sonication which causes plasma membrane vesicles to disrupt and reseal as smaller vesicles, consequently releasing the cytosolic interior into the solution.

In order to remove this additional contamination source, the PMV membranes can be pelleted by ultracentrifugation and the supernatant is removed.

The membrane pellet can also be rinsed and dispersed by sonication in a buffer solution, e.g., ammonium bicarbonate buffer.

The plasma membrane vesicles can also be filtered through a filter to produce a uniform size plasma membrane vesicle population.

The population of plasma membrane vesicles can be enriched for a given membrane protein by, for example, affinity purification or immuno-purification.

A variety of cells may be used to prepare plasma membrane vesicles. The cells or cell lines may grow attached to a surface or free in growth media. Cells can be from any organism, preferably from mammals, e.g., humans. In one embodiment, the cells used to make plasma membrane vesicles are cells associated with a disease state, e.g., cancer. In another embodiment, the cells are transformed or transfected to yield a protein of interest. In exemplary embodiments, the protein of interest is one or several membrane proteins, e.g., transmembrane proteins, or lipid-anchored proteins.

Nucleotide sequences encoding exogenous proteins may be introduced into cells to produce membrane vesicles using common molecular biology techniques known to those of skill in the art. The necessary elements for the transcription and translation of the inserted nucleotide sequences may be selected depending on the cell chosen, and may be readily accomplished by one of ordinary skill in the art. A reporter gene which facilitates the selection of cells transformed or transfected with a nucleotide acid sequence may also be incorporated in the microorganism. (See, e.g., Sambrook et al. Molecular Cloning A Laboratory Manual, 2nd edition, Cold Spring Harbor Laboratory Press, 1989, for transfection/transformation methods and selection of transcription and translation elements, and reporter genes). Sequences which encode exogenous proteins may generally be obtained from a variety of sources, including for example, depositories which contain plasmids encoding sequences including the American Type Culture Collection (ATCC, Rockville Md.), and the British Biotechnology Limited (Cowley, Oxford England).

A “transmembrane domain” spans a membrane, a “membrane anchoring domain” is positioned within, but does not traverse, a membrane. An “extracellular” or “displayed” domain is present on the exterior of a cell, or a plasma membrane vesicle, and is thus in contact with the external environment of the cell or plasma membrane vesicle.

A “eukaryote” is as the term is used in the art. A eukaryote may, by way of non-limiting example, be a fungus, a unicellular eukaryote, a plant or an animal. An animal may be a mammal, such as a rat, a mouse, a rabbit, a dog, a cat, a horse, a cow, a pig, a simian or a human.

A “eukaryotic membrane” is a membrane found in a eukaryote. A eukaryotic membrane may, by way of non-limiting example, be a cytoplasmic membrane, a nuclear membrane, a nucleolar membrane, a membrane of the endoplasmic reticulum (ER), a membrane of a Golgi body, a membrane of a lysosome a membrane of a peroxisome, a caveolar membrane, or an inner or outer membrane of a mitochondrion, chloroplast or plastid.

A “membrane protein” is a protein found in whole or in part in a membrane. Membrane proteins can have at least one membrane anchoring domain or at least one transmembrane domain.

An “expression vector” is an artificial nucleic acid molecule into which an exogenous nucleic acid molecule encoding a protein can be inserted in such a manner so as to be operably linked to appropriate expression sequences that direct the expression of the exogenous nucleic acid molecule.

By the term “operably linked” it is meant that the gene products encoded by the non-vector nucleic acid sequences are produced from an expression element in vivo.

An “expression construct” is an expression vector into which a nucleotide sequence of interest has been inserted in a manner so as to be positioned to be operably linked to the expression sequences present in the expression vector.

The instant invention provides method and compositions for the study and characterization of proteins that reside in or on a plasma membrane. Exemplary proteins that can be used in the methods and compositions of the invention are set forth below.

Membrane Proteins

Membrane proteins consist, in general, of two types, peripheral membrane proteins and integral membrane proteins.

Integral membrane proteins can span the two layers (or “leaflets”) of a lipid bilayer membrane. Thus, such proteins may have extracellular, transmembrane, and intracellular domains. Extracellular domains are exposed to the external environment of the cell, whereas intracellular domains face the cytosol of the cell. The portion of an integral membrane protein that traverses the membrane is the “transmembrane domain.” Transmembrane domains traverse the cell membrane often by one or more regions comprising typically 15 to 25 hydrophobic amino acids which are predicted to adopt an alpha-helical conformation.

Intergral membrane proteins are classified as bitopic or polytopic (Singer, (1990) Annu. Rev. Cell Biol. 6:247-96). Bitopic proteins span the membrane once while polytopic proteins contain multiple membrane-spanning segments.

A peripheral membrane protein is a membrane protein that is bound to the surface of the membrane and is not integrated into the hydrophobic layer of a membrane region. Peripheral membrane proteins do not span the membrane but instead are bound to the surface of a membrane, one layer of the lipid bilayer that forms a membrane, or the extracellular domain of an integral membrane protein.

The invention can be applied to any membrane protein, including but not limited to the following exemplary receptors and membrane proteins. The proteins include but are not limited to receptors (e.g., GPCRs, sphingolipid receptors, neurotransmitter receptors, sensory receptors, growth factor receptors, hormone receptors, chemokine receptors, cytokine receptors, immunological receptors, and compliment receptors, FC receptors), channels (e.g., potassium channels, sodium channels, calcium channels.), pores (e.g., nuclear pore proteins, water channels), ion and other pumps (e.g., calcium pumps, proton pumps), exchangers (e.g., sodium/potassium exchangers, sodium/hydrogen exchangers, potassium/hydrogen exchangers), electron transport proteins (e.g., cytochrome oxidase), enzymes and kinases (e.g., protein kinases, ATPases, GTPases, phosphatases, proteases.), structural/linker proteins (e.g., Caveolins, clathrin), adapter proteins (e.g., TRAD, TRAP, FAN), chemotactic/adhesion proteins (e.g., ICAM11, selectins, CD34, VCAM-1, LFA-1, VLA-1), and phospholipases such as PI-specific PLC and other phospholipiases.

Other membrane proteins are within the scope of the invention and include but are not limited to channels (e.g., potassium channels, sodium channels, calcium channels.), pores (e.g., nuclear pore proteins, water channels), ion and other pumps (e.g., calcium pumps, proton pumps), exchangers (e.g., sodium/potassium exchangers, sodium/hydrogen exchangers, potassium/hydrogen exchangers), electron transport proteins (e.g., cytochrome oxidase), enzymes and kinases (e.g., protein kinases, ATPases, GTPases, phosphatases, proteases.), structural/linker proteins (e.g., Caveolins, clathrin), adapter proteins (e.g., TRAD, TRAP, FAN),

Cellular Adhesion Molecules

Cellular adhesion molecules can be used in the methods and compositions of the invention. Exemplary cellular adhesion molecules include human rhinovirus receptor (ICAM-1), ICAM-2, ICAM-3, and PECAM-1, and chemotactic/adhesion proteins (e.g., selectins, CD34, VCAM-1, LFA-1, VLA-1) are within the scope of the invention. See also Alpin et al., “Signal Transduction and Signal Modulation by Cell Adhesion Receptors: The Role of Integrins, Cadherins, Immunoglobulin-Cell Adhesion Molecules, and Selectins”, Pharmacological Reviews, Vol. 50, No. 2.

In addition to the preceding non-limiting examples, the invention can be applied to the membrane proteins described in U.S. Pat. No. 6,335,018 (High molecular weight major outer membrane protein of moraxella); U.S. Pat. No. 6,264,954 (Haemophilus outer membrane protein); U.S. Pat. No. 6,197,543 (Human vesicle membrane protein-like proteins); U.S. Pat. No. 6,121,427 (Major outer membrane protein CD of branhamella); U.S. Pat. Nos. 6,083,743 and 6,013,514 (Haemophilus outer membrane protein); U.S. Pat. No. 6,004,562 (Outer membrane protein B1 of Moraxella catarrhalis); U.S. Pat. No. 5,863,764 (DNA encoding a human membrane protein); U.S. Pat. No. 5,861,283 (DNA encoding a limbic system-associated membrane protein); U.S. Pat. No. 5,824,321 (Cloned leptospira outer membrane protein); U.S. Pat. No. 5,821,085 (Nucleotide sequences of a T. pallidum rare outer membrane protein); U.S. Pat. No. 5,821,055 (Chlamydia major outer membrane protein); U.S. Pat. No. 5,808,024 (Nucleic acids encoding high molecular weight major outer membrane protein of moraxella); U.S. Pat. No. 5,770,714 (Chlamydia major outer membrane protein); U.S. Pat. No. 5,763,589 (Human membrane protein); U.S. Pat. No. 5,753,459 (Nucleotide sequences of T. pallidum rare outer membrane protein); U.S. Pat. No. 5,607,920 (Concanavalin a binding proteins and a 76 kD chondrocyte membrane protein (CMP) from chondrocytes and methods for obtaining same); and U.S. Pat. No. 5,503,992 (DNA encoding the 15 kD outer membrane protein of Haemophilus influenzae).

A variety of types and examples of transmembrane domains are known. The methods and compositions of the invention also pertain to the following types of transmembrane proteins.

Monotropic (“single pass”) domains, which traverse a membrane once, include by way of non-limiting example, those found in receptors for epidermal growth factor (EGF), receptors for tumor necrosis factor (TNF) and the like. Polytropic (“multipass”) proteins traverse a membrane two or more times. Non-limiting examples of polytropic proteins are as follows.

Biotropic (“2 passes”) membrane proteins include, but are not limited to: EnvZ of E. coli; the peroxisomal membrane protein Pex11-1p (Anton et al., ARF- and coatomer-mediated peroxisomal vesiculation, Cell Biochem Biophys 2000;32 Spring:27-36); pleitropic drug ABC transporters of S. cervisiae (Rogers et al., The pleitropic drug ABC transporters from Saccharomyces cerevisiae, J Mol Microbiol Biotechnol 2001 3:207-14); and human and rate urate transporters hUAT and rUAT (Lipkowitz et al., Functional reconstitution, membrane targeting, genomic structure, and chromosomal localization of a human urate transporter, J Clin Invest 2001 107:1103-15).

Tritropic (“3 pass”) membrane proteins include, but are not limited to: the ethylene receptor ETR1 of Arabidopsis; the Cauliflower Card Expression protein CC 1 (Palmer et al., A Brassica oleracea Gene Expressed in a Variety-Specific Manner May Encode a Novel Plant Transmembrane Receptor, Plant Cell Physiol 2001 42:404-413); and a splice variant of the mitochondrial membrane protein hMRS3/4 (Li et al., Characterization of a novel human putative mitochondrial transporter homologous to the yeast mitochondrial RNA splicing proteins 3 and 4, FEBS Lett 2001 494:79-84).

Tetraspanins or tetraspans are non-limiting examples of membrane proteins with four transmembrane domains. (Levy et al., J. Biol. Chem, 226:14597-14602, 1991; Tomlinson et al., J. 1 mmol. 23:136-40, 1993; and Barclay et al., (In) The Leucocyte antigen factbooks, Academic press, London, 1993). These proteins are collectively known as the transmembrane 4 superfamily (TM4) because they span the plasma membrane four times. The proteins known to belong to this family include, but are not limited to: mammalian antigen CD9 (MIC3), a protein involved in platelet activation and aggregation; mammalian leukocyte antigen CD37, expressed on B lymphocytes; mammalian leukocyte antigen CD53 (OX-44), which may be involved in growth regulation in hematopoietic cells; mammalian lysosomal membrane protein CD63 (Melanoma-associated antigen ME491; antigen AD1); mammalian antigen CD81 (cell surface protein TAPA-1), which may play an important role in the regulation of lymphoma cell growth; mammalian antigen CD82 (Protein R2; Antigen C33; Kangai 1 (KAI1)), which associates with CD4 or CD8 and delivers costimulatory signals for the TCR/CD3 pathway; mammalian antigen CD151 (SFA-1); Platelet-endothelial tetraspan antigen 3 (PETA-3); mammalian TM4SF2 (Cell surface glycoprotein A15; TALLA-1; MXS1); mammalian TM4SF3 (Tumor-associated antigen CO-029); mammalian TM4SF6 (Tspan-6; TM4-D); mammalian TM4SF7 (Novel antigen 2 (NAG-2); Tspan-4); mammalian Tspan-2; Mammalian Tspan-3 (TM4-A); mammalian Tetraspan NET-5; and Schistosoma mansoni and japonicum 23 Kd surface antigen (SM23/SJ23).

Non-limiting examples of membrane proteins with six transmembrane domains include the EBV integral membrane protein LMP-1, and a splice variant of the mitochondrial protein hMRS3/4 (Li et al., Characterization of a novel human putative mitochondrial transporter homologous to the yeast mitochondrial RNA splicing proteins 3 and 4, FEBS Lett Apr. 6, 2001; 494(1-2):79-84). Proteins with six transmembrane domains also include STEAP (six transmembrane epithelial antigens of the prostate) proteins (Afar et al., U.S. Pat. No. 6,329,503). The prototype member of the STEAP family, STEAP-1, appears to be a type IIIa membrane protein expressed predominantly in prostate cells in normal human tissues. Structurally, STEAP-1 is a 339 amino acid protein characterized by a molecular topology of six transmembrane domains and intracellular N- and C-termini, suggesting that it folds in a “serpentine” manner into three extracellular and two intracellular loops.

Hundreds of 7-pass membrane proteins are known. G-protein coupled receptors (GPCRs), including without limitation beta-adreno receptors, adrenergic receptors, EDG receptors, adenosine receptors, B receptors for kinins, angiotensin receptors, and opiod receptors are of particular interest. GPCRs are described in more detail elsewhere herein.

A non-limiting example of a protein with 9 transmembrane domains is Lipocalin-1 interacting membrane receptor (Wojnar et al., Molecular cloning of a novel Lipocalin-1 interacting human cell membrane receptor (LIMR) using phage-display, J Biol Chem 2001 3).

Proteins with both transmembrane and anchoring domains are known. For example, AMPA receptor subunits have transmembrane domains and one membrane-anchoring domain.

Lipid Constituents

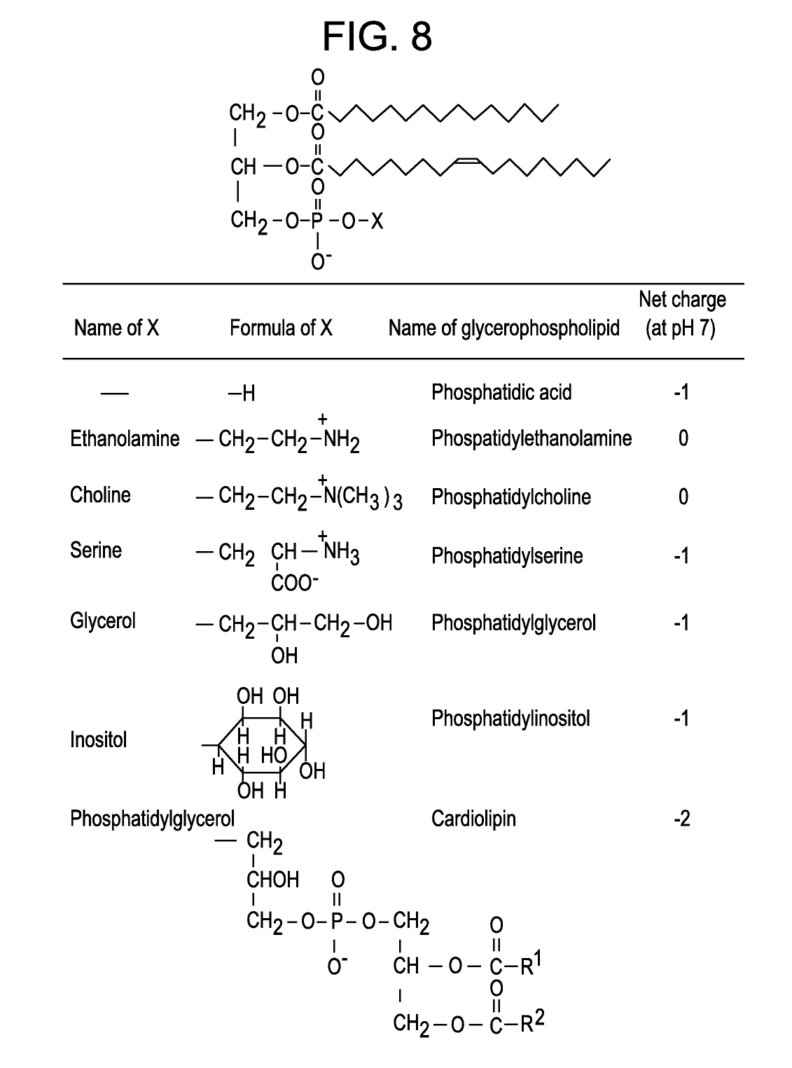

The most common lipids in the biomembrane are the 1,2-dialkylphosphoglycerides or phospholipids (Gennis, 1989). These include, for example, phosphatidyl choline (PC), phosphatidyl ethanolamine (PE), phosphatidyl serine (PS) and phosphatidyl glycerol (PG). The structure of these phospholipids are summarized in FIG. 8.

The phospholipid structures consist of two alkyl chains or fatty acids that are bound to a common glycerol molecule by ester bonds. The third hydroxyl group is linked to a phosphate molecule, which is connected to the various head-groups of the lipids. The alkyl chains or hydrocarbon tails varies both in lengths (from 14-24 carbon atoms) and degree of saturation, which together dictates such fundamental properties as permeability and fluidity of the membrane, for example. The head-group on the other hand contains information of the charge of the molecule, which also affects the properties and functionality of the membrane. The most common phospholipid is phosphatidyl choline, whose head-group consists of a tertiary amine. This type of lipid is zwitterionic, which means that the structure bears a net charge of zero at neutral pH values. This occurs by balancing the charges that is located on the phosphate (negative charge) and the tertiary amine (positive charge). Phosphatidyl serine on the other hand obtains a net charge of −1 at neutral pH, since it contains both a carboxyl group (negative charge) and an amine on the head-group.

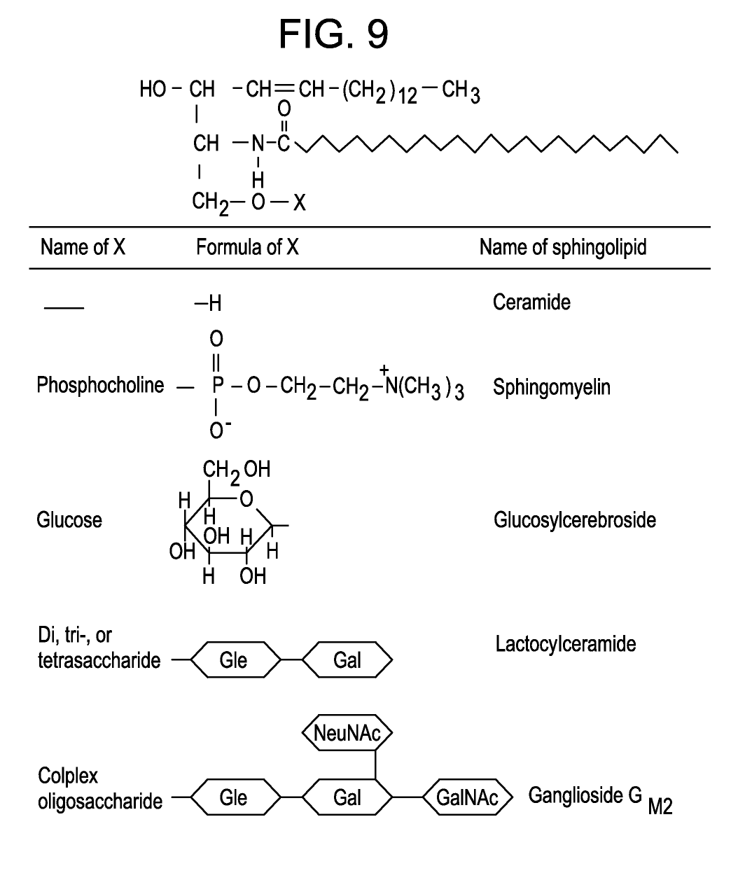

Sphingolipids are also common in biomembranes and consist of one molecule of the long-chain amino alcohol sphingosine or one of its derivatives, one molecule of a long-chain fatty acid and a polar head alcohol, which sometimes have a phosphodiester linkage. The sphingolipids can also be sub-divided into three groups, sphingomyelins, glycolipids and gangliosides (FIG. 9). Sphingomyelins have similarities with phosphatidylcholines in properties and structure and are present in plasma membranes of animal cells. The glycolipids and gangliosides are also found in animal cell plasma membranes, with a high presence in neural tissues, such as the brain, and have sugar units attached to their polar head groups.

The sterols of which cholesterol is the most common in animal tissues have a polar head-group and a non-polar hydrocarbon body with a length about the same as a 16-carbon fatty acid in its extended form. Sterols are often precursors for molecules with specific biological functions, such as the bile acids that act as detergents in the intestine or steroid hormones.

Many different solvents can be used to dissolve lipids, however they are only suitable for extracting lipids from cellular material and tissues if they can break the associations between the lipids and other cellular constituents, such as proteins and polysaccharides. Ideally, the solvent or solvent mixture should be fairly polar in order to release all lipids from their association with cell membranes or with lipoproteins. The extracting solvent may also prevent to some extent enzymatic hydrolysis.

Some structural features of lipids, such as the hydrophobic hydrocarbon chains of the fatty acid or other aliphatic moieties and any polar functional groups such as phosphate or sugar residues, which are markedly hydrophilic control the solubility of the lipids in organic solvents. Lipids that lack polar groups, for example triacylglycerols or cholesterol esters, are soluble in hydrocarbons such as hexane, toluene or cyclohexane and in more polar solvents such as diethyl ether or chloroform for example. These are rather insoluble polar solvents such as methanol though. Polar lipids, such as phospholipids and glycosphingolipids, unless solubilized by other types of lipids, are only slightly soluble in hydrocarbons, but they are easily dissolved in more polar solvents like methanol, ethanol or chloroform. The high dielectric constants and polarity of these solvents overcomes the ion-dipole interactions and hydrogen bonding.

Most complex lipids are slightly soluble in water and at least form micellar solutions, and lipids such as gangliosides, polyphosphoinositides, lysophospholipids, acyl-carnitines and coenzyme A esters are especially soluble. Pure solvents are usually not useful as a general purpose lipid extractants. A mixture of solvents is more useful and one of the most widely used mixtures is chloroform and methanol at a ratio of 2:1 (v/v). This mixture will extract lipids from tissues (animal, plant and bacteria) more thoroughly than other simple solvent combinations. In some other studies, dichloromethane (DCM)-methanol (2:1, v/v) was found to be as effective as the chloroform-methanol mixture and the lower toxicity of dichloromethane can be an advantage.

Mixtures of propan-2-ol and hexane (3:2, v/v) have also been used for the extraction of lipids from animal tissues and this mixture has a lower toxicity. Methanol-hexane (1:1, v/v) has been used for extraction of lipids from leaf tissue. Hexane-ethanol (5:2, v/v) has been used for the extraction of ubiquinone and heptane-ethanol with the surfactant sodium dodecyl sulphate added has been recommended for determining vitamin E/lipid ratios in animal tissues. Other mixtures that has been tested for lipid solubility are toluene-ethanol, benzene-ethanol, benzene-methanol, propan-2-ol-benzene-water (2:2:1, v/v), butan-1-ol saturated with water, hexan-2-ol, and butan-1-ol-diisopropyl ether (2:3, v/v). Diethyl ether and chloroform alone are also good solvents for lipids, however not so good att extracting lipids from tissues for example. When they are used to extract plant tissues, these solvents also enhance the action of phospholipase D unfortunately, as does butan-1-ol. Propan-1-ol and propan-2-ol strongly inhibit this reaction and the latter, which has the lower boiling point, has been recommended for use with plant tissues, as a preliminary extractant especially.

Acetone can dissolve simple lipids and glycolipids, however it will not dissolve phospholipids readily and it is actually often used to precipitate phospholipids from solution in other solvents. Supercritical fluids have also been tested for lipid extraction purposes and results indicate that this procedure will work for simple lipids.

Methods of Using the Plasma Membrane Vesicles of the Invention

The plasma membrane vesicles of the invention can be used for a multitude of purposes. For example, the plasma membrane vesicles can be used to study membrane proteins that are not soluble outside of the membrane. They can also be used to screen for modulators of membrane proteins, or can be used in a reverse screen to identify membrane proteins that bind a known ligand. In other embodiments, the plasma membrane vesicles of the invention can be used to study the protein expression pattern of cells relative to each other, or of similar cells at different points in time, e.g., upon contact with a ligand or upon converting to a disease state, e.g., cancerous.

In an exemplified embodiment, the plasma membrane vesicles of the invention are used to study the expression patterns of cell surface proteins. In one embodiment, plasma membrane vesicles are prepared from cells of interest at the desired time by using the methods described herein.

The plasma membrane vesicles are contacted with a protease, for example, trypsin or chymotrypsin, and the resulting protein fragments are identified. Polypeptide fragments can be identified by any of a number of art recognized methods.

Enzymatic digestion may be performed in-solution, as well as after plasma membrane vesicles are immobilized in a flowcell. For in-solution digestion, protease can be added to the processed plasma membrane vesicles solution, and the peptides can be separated from the membranes by size filtration.

Polypeptide fragments can be analyzed by mass spectroscopy. In an exemplified embodiment, the fragments are analyzed using LC MS/MS. Liquid chromatography separates the individual components contained within a sample so that they may be identified. The separated components may be fed into a mass spectrometer for further analysis in order to determine their identity. Systems with two mass spectrometer stages are referred to as LC-MS/MS systems. A mass spectrometer takes a sample as input and ionizes the sample to create either positive or negative ions. A number of different ionization methods may be used including the use of electrospray ionization. The ions are then separated by the mass to charge ratio in a first stage separation, commonly referred to as MS1. The mass separation may be accomplished by a number of means including the use of magnets which divert the ions to differing degrees based upon the weight of the ions. The separated ions then travel into a collision cell where they come in contact with a collision gas or other substance which interacts with the ions. The reacted ions then undergo a second stage of mass separation commonly referred to as MS2.

The separated ions are analyzed at the end of the mass spectrometry stage (or stages). The analysis graphs the intensity of the signal of the ions versus the mass-to-charge ratio of the ion in a graph referred to as a mass spectrum. The analysis of the mass spectrum gives both the masses of the ions reaching the detector and the relative abundances. The abundances are obtained from the intensity of the signal. The combination of liquid chromatography with mass spectrometry may be used to identify chemical substances such as metabolites. When a molecule collides with the collision gas covalent bonds often break, resulting in an array of charged fragments. The mass spectrometer measures the masses of the fragments which may then be analyzed to determine the structure and/or composition of the original molecule. This feature is significantly enhanced from nominal mass MS when using a mass spectrometer capable of accurate mass measurements e.g. hybrid quadrupole orthoganol TOF instrument or FTICR, allowing analyte elemental composition information to be derived. This information may be used to isolate a particular substance in a sample.

In an exemplified embodiment, the plasma membrane vesicles of the invention are used to study the lipid composition of the plasma membrane, by extraction of the lipid components from the purified plasma membrane vesicles.

In another embodiment, the presence of absence of a particular membrane protein can be evaluated by analyzing the polypeptide fragments by immunohistochemistry. Accordingly, in another embodiment, an immunoassay can be used to detect and analyze peptide fragments. This method comprises: (a) providing an antibody that specifically binds to a peptide of interest; (b) contacting a sample with the antibody; and (c) detecting the presence of a complex of the antibody bound to the peptide in the sample.

To prepare an antibody that specifically binds to a peptide, purified peptides or their nucleic acid sequences can be used. Nucleic acid and amino acid sequences for peptides can be obtained by further characterization of these markers. The molecular weights of digestion fragments from each marker can be used to search the databases, such as SwissProt database, for sequences that will match the molecular weights of digestion fragments generated by various enzymes. Using this method, the nucleic acid and amino acid sequences of other peptides can be identified if these markers are known proteins in the databases.

Assays

Plasma membrane vesicles could also be used in manual, semi-automated, automated and/or robotic assays for the identification of compounds that interact with a membrane protein contained in the plasma membrane vesicle.

Plasma membrane vesicles can be used in assays for screening pharmacological agents. By way of non-limiting example, the plasma membrane vesicles provide an environment for the expression of membrane proteins and studies and for the identification of modulators.

Another technique for assessing protein expression involves the use of western blots. Antibodies directed to various expressed proteins of interest have been generated and many are commercially available. Techniques for generating antibodies to proteins or polypeptides derived therefrom are known in the art (see, e.g., Cooper et al., Section III of Chapter 11 in: Short Protocols in Molecular Biology, 2nd Ed., Ausubel et al., eds., John Wiley and Sons, New York, 1992, pages 11-22 to 11-46). Standard western blot protocols, which may be used to show protein expression from the expression vectors in plasma membrane vesicles and other expression systems, are known in the art. (see, e.g., Winston et al., Unit 10.7 of Chapter 10 in: Short Protocols in Molecular Biology, 2nd Ed., Ausubel et al., eds., John Wiley and Sons, New York, 1992, pages 10-32 to 10-35).

High-Throughput Screening (HTS)

HTS typically uses automated assays to search through large numbers of compounds for a desired activity. Typically HTS assays are used to find new drugs by screening for chemicals that act on a particular enzyme or molecule. For example, if a chemical inactivates an enzyme it might prove to be effective in preventing a process in a cell that causes a disease. High throughput methods enable researchers to try out thousands of different chemicals against each target very quickly using robotic handling systems and automated analysis of results.

As used herein, “high throughput screening” or “HTS” refers to the rapid in vitro screening of large numbers of compounds (libraries); generally tens to hundreds of thousands of compounds, using robotic screening assays. Ultra high-throughput Screening (uHTS) generally refers to the high-throughput screening accelerated to greater than 100,000 tests per day.

Screening assays may include controls for purposes of calibration and confirmation of proper manipulation of the components of the assay. Blank wells that contain all of the reactants but no member of the chemical library are usually included. As another example, a known inhibitor (or activator) of an enzyme for which modulators are sought, can be incubated with one sample of the assay, and the resulting decrease (or increase) in the enzyme activity determined according to the methods herein. It will be appreciated that modulators can also be combined with the enzyme activators or inhibitors to find modulators which inhibit the enzyme activation or repression that is otherwise caused by the presence of the known the enzyme modulator. Similarly, when ligands to a sphingolipid target are sought, known ligands of the target can be present in control/calibration assay wells.

The plasma membrane vesicles of the invention are readily adaptable for use in high-throughput screening assays for screening candidate compounds to identify those which have a desired activity, e.g., blocking the binding of a ligand to a receptor. The compounds thus identified can serve as conventional “lead compounds” or can themselves be used as therapeutic agents.

The methods of screening of the invention comprise using screening assays to identify, from a library of diverse molecules, one or more compounds' having a desired activity. A “screening assay” is a selective assay designed to identify, isolate, and/or determine the structure of, compounds within a collection that have a preselected activity. By “identifying” it is meant that a compound having a desirable activity is isolated, its chemical structure is determined (including without limitation determining the nucleotide and amino acid sequences of nucleic acids and polypeptides, respectively) the structure of and, additionally or alternatively, purifying compounds having the screened activity). Biochemical and biological assays are designed to test for activity in a broad range of systems ranging from protein-protein interactions, enzyme catalysis, small molecule-protein binding, agonists and antagonists, to cellular functions. Such assays include automated, semi-automated assays and HTS (high throughput screening) assays.

In HTS methods, many discrete compounds are preferably tested in parallel by robotic, automatic or semi-automatic methods so that large numbers of test compounds are screened for a desired activity simultaneously or nearly simultaneously. It is possible to assay and screen up to about 6,000 to 20,000, and even up to about 100,000 to 1,000,000 different compounds a day using the integrated systems of the invention.

High throughput competitive inhibition assays are designed to identify agents that inhibit a specific target protein. Plasma membrane vesicles that express and/or display a specific membrane protein could be used in all types of competitive inhibition assays.

Plasma membrane vesicles of this invention are used in “functional screening HTS assays”. Functional screening assays are defined as assays that provide information about the function of a specific target protein. Functional assays screen agents against specific target proteins to identify agents that either act as antagonist or as an agonist against the protein. Functional assays require that the target protein be in an environment that allows it to carry out its natural function. Such functions include, but are not limited to G-proteins coupling with a GPCR, enzymatic activity such as phosphorlyation or proteolysis, protein-protein interaction, and transport of molecules and ions.

Functional assays screen agents against proteins which are capable of natural function. Target proteins used in functional studies must carry out a function that is measurable. Examples of protein functions that are measurable include but are not limited to the use of Fluorescent Resonance Energy Transfer (FRET) to measure the G-protein coupling to a GPCR (Ruiz-Velasco et al., Functional expression and FRET analysis of green fluorescent proteins fused to G-protein subunits in rat sympathetic neurons, J. Physiol. 537:679-692, 2001; Janetopoulos et al., Receptor-mediated activation of heterotrimeric G-proteins in living cells, Science 291:2408-2411, 2001); Bioluminescence Resonance Energy Transfer (BRET) to assay for functional ligand induced G-protein coupling to a target GPCR (Menard, L. Bioluminescence Resonance Energy Transfer (BRET): A powerful platform to study G-protein coupled receptors (GPCR) activity in intact cells, Assay Development, Nov. 28-30, 2001), the use of fluorescent substrates to measure the enzymatic activity of proteases (Grant, Designing biochemical assays for proteases using fluorogenic substrates, Assay Development, Nov. 28-30, 2001); and the determination of ion channel function via the use of voltage sensitive dyes (Andrews et al, Correlated measurements of free and total intracellular calcium concentration in central nervous system neurons, Microsc Res Tech. 46:370-379, 1999).

One non-limiting example of high throughput functional screening assay using plasma membrane vesicles for the functional coupling of GPCRs to their respective G-protein. Upon ligand binding, voltage polarization, ion binding, light interaction and other stimulatory events activate GPCRs and cause them to couple to their respective G-protein. In a plasma membrane vesicle, both the GPCR and its respective G-proteins can be simultaneously expressed. Upon activation of the GPCR, the coupling event will occur in the plasma membrane vesicle. Thus by detecting this coupling in the plasma membrane vesicle, one could screen for agents that bind GPCRs to identify antagonists and agonists. The antagonists are identified using inhibition assays that detect the inhibition of function of the GPCR. Thus the agent interacts with the GPCR in a way that it inhibits the GPCR from being activated. The agonists are identified by screening for agents that activate the GPCR in the absence of the natural activator.

Another non-limiting example of plasma membrane vesicles used for functional assays involves the screening of agonists/antagonists for ion channels. One example is the calcium channel, SCaMPER, encoded on a poycistronic episomal plasmid, which also encodes for a luminescent soluble protein, aequorin. In this assay, the plasma membrane vesicles will contain aequorin proteins in its cytoplasm and SCaMPER proteins expressed on the plasma membrane vesicles. Thus upon activation of SCaMPER by its ligand, SPC, or by an analog thereof, calcium will flow into the plasma membrane vesicle and will be bound by the aequorin which will luminescence. Thus a detection signal for the functional activation of the calcium channel is obtained.

Plasma membrane vesicles can also be employed for expression of target proteins and the preparation of membrane preparations for use in screening assays. Such proteins include but are not limited to receptors (e.g., GPCRs, receptors, neurotransmitter receptors, sensory receptors, growth factor receptors, hormone receptors, chemokine receptors, cytokine receptors, immunological receptors, and compliment receptors, FC receptors), channels (e.g., potassium channels, sodium channels, calcium channels.), pores (e.g., nuclear pore proteins, water channels), ion and other pumps (e.g., calcium pumps, proton pumps), exchangers (e.g., sodium/potassium exchangers, sodium/hydrogen exchangers, potassium/hydrogen exchangers), electron transport proteins (e.g., cytochrome oxidase), enzymes and kinases (e.g., protein kinases, ATPases, GTPases, phosphatases, proteases.), structural/linker proteins (e.g., Caveolins, clathrin), adapter proteins (e.g., TRAD, TRAP, FAN), chemotactic/adhesion proteins (e.g., ICAM11, selectins, CD34, VCAM-1, LFA-1, VLA-1), and chimeric/fusion proteins (e.g., proteins in which a normally soluble protein is attached to a transmembrane region of another protein). In such assays the membrane preparations are used to screen for agents that are either antagonists or agonists.

Chemical Libraries

Developments in combinatorial chemistry allow the rapid and economical synthesis of hundreds to thousands of discrete compounds. These compounds are typically arrayed in moderate-sized libraries of small organic molecules designed for efficient screening. Combinatorial methods, can be used to generate unbiased libraries suitable for the identification of novel inhibitors. In addition, smaller, less diverse libraries can be generated that are descended from a single parent compound with a previously determined biological activity. In either case, the lack of efficient screening systems to specifically target therapeutically relevant biological molecules produced by combinational chemistry such as inhibitors of important enzymes hampers the optimal use of these resources.

A combinatorial chemical library is a collection of diverse chemical compounds generated by either chemical synthesis or biological synthesis, by combining a number of chemical “building blocks,” such as reagents. For example, a linear combinatorial chemical library, such as a polypeptide library, is formed by combining a set of chemical building blocks (amino acids) in a large number of combinations, and potentially in every possible way, for a given compound length (i.e., the number of amino acids in a polypeptide compound). Millions of chemical compounds can be synthesized through such combinatorial mixing of chemical building blocks.

A “library” may comprise from 2 to 50,000,000 diverse member compounds. Preferably, a library comprises at least 48 diverse compounds, preferably 96 or more diverse compounds, more preferably 384 or more diverse compounds, more preferably, 10,000 or more diverse compounds, preferably more than 100,000 diverse members and most preferably more than 1,000,000 diverse member compounds. By “diverse” it is meant that greater than 50% of the compounds in a library have chemical structures that are not identical to any other member of the library. Preferably, greater than 75% of the compounds in a library have chemical structures that are not identical to any other member of the collection, more preferably greater than 90% and most preferably greater than about 99%.

The preparation of combinatorial chemical libraries is well known to those of skill in the art. For reviews, see Thompson et al., Synthesis and application of small molecule libraries, Chem Rev 96:555-600, 1996; Kenan et al., Exploring molecular diversity with combinatorial shape libraries, Trends Biochem Sci 19:57-64, 1994; Janda, Tagged versus untagged libraries: methods for the generation and screening of combinatorial chemical libraries, Proc Natl Acad Sci USA. 91:10779-85, 1994; Lebl et al., One-bead-one-structure combinatorial libraries, Biopolymers 37:177-98, 1995; Eichler et al., Peptide, peptidomimetic, and organic synthetic combinatorial libraries, Med Res Rev. 15:481-96, 1995; Chabala, Solid-phase combinatorial chemistry and novel tagging methods for identifying leads, Curr Opin Biotechnol. 6:632-9, 1995; Dolle, Discovery of enzyme inhibitors through combinatorial chemistry, Mol Divers. 2:223-36, 1997; Fauchere et al., Peptide and nonpeptide lead discovery using robotically synthesized soluble libraries, Can J Physiol Pharmacol. 75:683-9, 1997; Eichler et al., Generation and utilization of synthetic combinatorial libraries, Mol Med Today 1: 174-80, 1995; and Kay et al., Identification of enzyme inhibitors from phage-displayed combinatorial peptide libraries, Comb Chem High Throughput Screen 4:535-43, 2001.

Such combinatorial chemical libraries include, but are not limited to, peptide libraries (see, e.g., U.S. Pat. No. 5,010,175, Furka, Int. J. Pept. Prot. Res., 37:487-493 (1991) and Houghton, et al., Nature, 354:84-88 1991). Other chemistries for generating chemical diversity libraries can also be used. Such chemistries include, but are not limited to, peptoids (PCT Publication No. WO 91/19735); encoded peptides (PCT Publication WO 93/20242); random bio-oligomers (PCT Publication No. WO 92/00091); benzodiazepines (U.S. Pat. No. 5,288,514); diversomers, such as hydantoins, benzodiazepines and dipeptides (Hobbs, et al., Proc. Nat. Acad. Sci. USA, 90:6909-6913 1993); vinylogous polypeptides (Hagihara, et al., J. Amer. Chem. Soc. 114:6568 1992); nonpeptidal peptidomimetics with .beta.-D-glucose scaffolding (Hirschmann, et al., J. Amer. Chem. Soc., 114:9217-9218 1992); analogous organic syntheses of small compound libraries (Chen, et al., J. Amer. Chem. Soc., 116:2661 1994); oligocarbamates (Cho, et al., Science, 261:1303 1993); and/or peptidyl phosphonates (Campbell, et al., J. Org. Chem. 59:658 1994); nucleic acid libraries (see, Ausubel, Berger and Sambrook, all supra); peptide nucleic acid libraries (see, e.g., U.S. Pat. No. 5,539,083); antibody libraries (see, e.g., Vaughn, et al., Nature Biotechnology, 14(3):309-314 (1996) and PCT/US96/10287); carbohydrate libraries (see, e.g., Liang, et al., Science, 274:1520-1522 (1996) and U.S. Pat. No. 5,593,853); small organic molecule libraries (see, e.g., benzodiazepines, Baum C&E News, January 18, page 33 (1993); isoprenoids (U.S. Pat. No. 5,569,588); thiazolidinones and metathiazanones (U.S. Pat. No. 5,549,974); pyrrolidines (U.S. Pat. Nos. 5,525,735 and 5,519,134); morpholino compounds (U.S. Pat. No. 5,506,337); benzodiazepines (U.S. Pat. No. 5,288,514); and the like.

Reverse Screening

In one aspect, the invention provides methods for screening libraries of plasma membrane vesicles in which each plasma membrane vesicle comprises an expression element that encodes a few, preferably one, membrane protein in order to identify a membrane protein that interacts with a preselected compound. By way of non-limiting example, sequences encoding membrane proteins, fusion proteins, or cytoplasmic proteins are cloned into an expression vector, either by “shotgun” cloning or by directed cloning, e.g., by screening or selecting for cDNA clones, or by PCR amplification of DNA fragments, that encode a protein using one or more oligonucleotides encoding a highly conserved region of a protein family. For a non-limiting example of such techniques, see Krautwurst, D., et al. 1998. Identification of ligands for olfactory receptors by functional expression of a receptor library. Cell 95:917-926. By way of non-limiting example, a plasma membrane vesicle expressing a receptor binds a preselected ligand, which may be a drug. Various assays for receptor binding, enzymatic activity, and channeling events are known in the art and may include detectable compounds; in the case of binding assays, competition assays may also be used (Masimirembwa, C. M., et al. 2001. In vitro high throughput screening of compounds for favorable metabolic properties in drug discovery. Comb. Chem. High Throughput Screen. 4:245-263; Mattheakis, L. C., and A. Saychenko. 2001. Assay technologies for screening ion channel targets. Curr. Opin. Drug Discov. Devel. 4:124-134; Numann, R., and P. A. Negulescu. 2001. High-throughput screening strategies for cardiac ion channels. Trends Cardiovasc. Med. 11:54-59; Le Poul, E., et al. 2002. Adaptation of aequorin functional assay to high throughput screening. J. Biomol. Screen. 7:57-65; and Graham, D. L., et al. 2001. Application of beta-galactosidase enzyme complementation technology as a high throughput screening format for antagonists of the epidermal growth factor receptor. J. Biomol. Screen. 6:401-411).

Once a plasma membrane vesicle has been identified by an assay and isolated, the membrane protein is identified. The ligands, antagonists and agonists may be used as lead compounds and/or drugs to treat diseases in which the membrane protein plays a role. In particular, when the preselected ligand is a drug, diseases for which that drug is therapeutic are expected to be treated using the novel ligands, antagonists and agonists, or drugs and prodrugs developed therefrom.

Determining the Structures of Membrane Proteins

Three-dimensional (3D) structures of proteins may be used for drug discovery. However, membrane proteins present challenging problems for 3D structure determination. Muller, Towards 3D structures of G protein-coupled receptors: a multidisciplinary approach. (Review), Curr Med Chem 2000 pp.861-88; Levy et al., Two-dimensional crystallization on lipid layer: A successful approach for membrane proteins, J Struct Biol 1999 127, 44-52. Although the three-dimensional structures of hundreds of different folds of globular proteins have been determined, fewer than 20 different integral membrane protein structures have been determined. There are many reasons for this. Extracting membrane proteins from the membrane can easily disrupt their native structure, and membrane proteins are notoriously difficult to crystallize.

Some membrane proteins readily form two-dimensional crystals in membranes and can be used for structure determination using electron diffraction spectroscopy (ED) instead of x-ray crystallography.

Nuclear magnetic resonance (NMR) is an alternative method for determining membrane protein structure, but most membrane proteins are too large for high-resolution NMR at the present state of the art. Furthermore, membrane proteins require special conditions for NMR, e.g. deuterated lipids must be used to avoid confusing the signal of the protein protons with the noise of membrane lipid protons.

The plasma membrane vesicles of the instant invention may be used to determine the structures of membrane proteins that are not soluble when removed from the membrane.

Differential Protein Expression Profiling Analysis

The present invention also provide methods for identifying differentially expressed proteins by protein expression profiling analysis. Protein expression profiles can be generated by any method permitting the resolution and detection of proteins from a sample from a population of plasma membrane vesicles made from a cell or cell line. Methods with higher resolving power are generally preferred, as increased resolution can permit the analysis of greater numbers of individual proteins, increasing the power and usefulness of the profile. A sample can be pre-treated to remove abundant proteins from a sample, such as by immunodepletion, prior to protein separation and detection, as the presence of an abundant protein may mask more subtle changes in expression of other proteins, particularly for low-abundance proteins. A sample can also be subjected to one or more procedures to reduce the complexity of the sample. For example, chromatography can be used to fractionate a sample; each fraction would have a reduced complexity, facilitating the analysis of the proteins within the fractions.

Three useful methods for simultaneously resolving and detecting several proteins include array-based methods; mass-spectrometry based methods; and two-dimensional gel electrophoresis based methods.

Protein arrays generally involve a significant number of different protein capture reagents, such as antibodies or antibody variable regions, each immobilized at a different location on a solid support. Such arrays are available, for example, from Sigma-Aldrich as part of their Panorama line of arrays. The array is exposed to a protein sample and the capture reagents selectively capture the specific protein targets. The captured proteins are detected by detection of a label. For example, the proteins can be labeled before exposure to the array; detection of a label at a particular location on the array indicates the detection of the corresponding protein. If the array is not saturated, the amount of label detected may correlate with the concentration or amount of the protein in the sample. Captured proteins can also be detected by subsequent exposure to a second capture reagent, which can itself be labeled or otherwise detected, as in a sandwich immunoassay format.

Mass spectrometry-based methods include, for example, matrix-assisted laser desorption/ionization (MALDI), Liquid Chromatography/Mass Spectrometry/Mass Spectrometry (LC-MS/MS) and surface enhanced laser desorption/ionization (SELDI) techniques. For example, a protein profile can be generated using electrospray ionization and MALDI. SELDI, as described, for example, in U.S. Pat. No. 6,225,047, incorporates a retention surface on a mass spectrometry chip. A subset of proteins in a protein sample are retained on the surface, reducing the complexity of the mixture. Subsequent time-of-flight mass spectrometry generates a “fingerprint” of the retained proteins.

In methods involving two-dimensional gel electrophoresis, proteins in a sample are generally separated in a first dimension by isoelectric focusing and in a second dimension by molecular weight during SDS-PAGE. By virtue of the two dimensions of resolution, hundreds or thousands of proteins can be simultaneously resolved and analyzed. The proteins are detected by application of a stain, such as a silver stain, or by the presence of a label on the proteins, such as a Cy2, Cy3, or Cy5 dye. To identify a protein, a gel spot can be cut out and in-gel tryptic digestion performed. The tryptic digest can be analyzed by mass spectrometry, such as MALDI. The resulting mass spectrum of peptides, the peptide mass fingerprint or PMF, is searched against a sequence database. The PMF is compared to the masses of all theoretical tryptic peptides generated in silico by the search program. Programs such as Prospector, Sequest, and MasCot (Matrix Science, Ltd., London, UK) can be used for the database searching. For example, MasCot produces a statistically-based Mowse score indicates if any matches are significant or not. MS/MS can be used to increase the likelihood of getting a database match. CID-MS/MS (collision induced dissociation of tandem MS) of peptides can be used to give a spectrum of fragment ions that contain information about the amino acid sequence. Adding this information to a peptide mass fingerprint allows Mascot to increase the statistical significance of a match. It is also possible in some cases to identify a protein by submitting only a raw MS/MS spectrum of a single peptide.

### Reconstitution of Membrane Proteins in Extracted Lipids

In situ study of biological membranes is difficult due to the vast complexity of lipids and proteins in the membrane. In many instances it is therefore vital to purify membrane proteins from the native membrane and re-insert the membrane protein into an artificial membrane. This process is referred to as reconstitution. Reconstitution is most often necessary in order for the membrane proteins to have intact functionality, which occurs when the membrane protein is correctly folded and inserted into a lipid bilayer. There are a plethora of methods to reconstitute membrane proteins and there seem to be no general protocol for this process, however the methods usually include one or several of: mechanical means (sonication or shearing of the membrane proteins together with lipid), freeze-thaw, organic solvents and detergents. Detergents are the most common and widely used for reconstitution purposes and most efforts goes into finding the right conditions that preserves the activity of the membrane proteins throughout the process. The orientation and insertion of the membrane proteins, the morphology and size of the reconstituted proteoliposomes and their permeability are also important factors.

Reconstitution normally proceed via co-micellization of the pure membrane protein together with excess of (phospho-)lipids and appropriate detergent(s) to create a solution of mixed lipid-protein-detergent and lipid-detergent micelles. The detergent is then removed from the micellar solution, which results in the formation of closed lipid bilayers with incorporated membrane proteins. Many methods and protocols exist in the literature and they differ mainly in the techniques to remove the detergent.

In several papers it has been noted that membrane proteins are heavily influenced by their surrounding lipid environment. In some cases, certain specific lipids have been shown to be essential for some membrane proteins functionality. Also, bilayer properties can influence the membrane proteins. For example, a miss match of the hydrophobic length of the protein and the lipid bilayer can strongly influence the functionality of the membrane protein. The elastic properties and the bilayer, which includes curvature energy and lateral pressure may also influence the membrane proteins.

The method described herein thus enables the purification of plasma membrane lipids from specific cell types for further use in reconstitution experiments. The benefits arise from the fact that one retains the cell-specific lipid components of the plasma membrane. When performing reconstitution of a membrane protein emanating from a plasma membrane of a specific cell line the reconstitution can be performed with the same lipids as in the native membrane.

### EXAMPLES

It should be appreciated that the invention should not be construed to be limited to the examples that are now described; rather, the invention should be construed to include any and all applications provided herein and all equivalent variations within the skill of the ordinary artisan.

### Example 1

### Production of High-Purity Plasma Membrane Vesicles (PMVs)

The production of high-purity plasma membrane vesicles consists of four steps:

1. Formation of PMVs by addition of PMV-forming agents to a cell culture

2. Release of PMVs from cell culture

3. Purification of PMVs by density gradient centrifugation

4. Purification of PMVs by dialysis

Steps 1-2 yields a crude polydisperse PMV fraction with sizes of PMVs ranging up to about ten micrometers in diameter. Such PMVs can find great use in several structural and functional assays. Examples of such assays include ion channel function, G-Protein function, adhesion protein function, and many more.

Adding steps 3 and 4 yield a cell-free ultrapure polydisperse PMV fraction with sizes of PMVs ranging up to about ten micrometers in diameter that can be utilized in several structural and functional assays, including proteomic assays to screen for protein expression, and target identification.

Formation of PMVs by addition of PMV-forming agents to an adherent cell culture. In order to produce and purify PMVs in high yields, a sufficient amount of cells has to be cultured. The method is scalable and works well with micropreps where even single PMVs can be collected from single cells up to large batch preparations where PMVs can be collected from hundreds of millions of cells. In addition to adherent cells, suspended cells can be used as well, and follow the same procedure as here described for adherent cells.

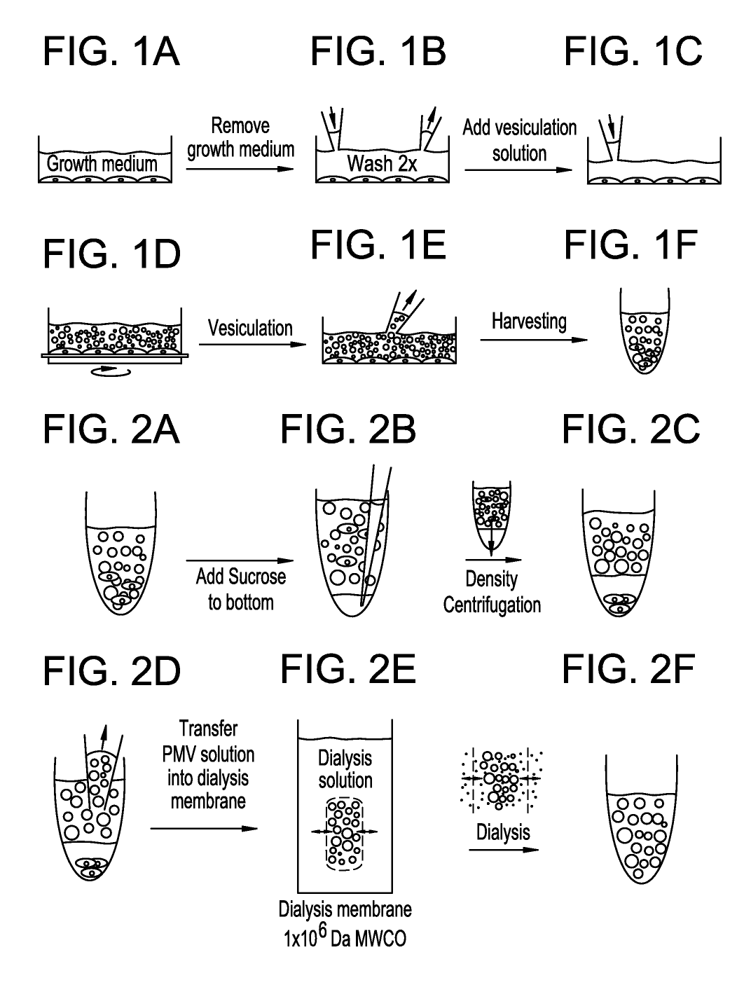

Adherent cells are grown to ˜80% confluency to obtain ˜15×10⁶ cells (See FIG. 1A). The cell layer is then thoroughly washed, using a buffer solution containing 10 mM HEPES and 140 mM NaCl to completely remove the culture medium, as serum proteins would pose a source of contamination in proteomic analysis (FIG. 1B).

Vesiculation is then induced by adding vesiculation solution, containing 2 mM dithiothreitol (DTT) and 25 mM formaldehyde (FA) directly to the culture flask (FIG. 1C). Using FA and DTT, PMVs start to develop after ˜15 min.

After ˜30 min of incubation, the flask is mechanically agitated via slow shaking (FIG. 1D). This makes the vesicles bud off from the cell layer resulting in a free-floating PMV suspension. The agitation can be performed manually i.e. shaking the flask or by use of a mechanical laboratory shaker or alternatively by use of ultrasonication.

Vesiculation was performed at 37° C. and was allowed to continue over a time period from 30 min up to several hours, in order to maximize the yield. The formed vesicles are transferred from the cell culture dish using a pipette (FIG. 1E) to an Eppendorf vial (FIG. 1F).

A single NG108-15 cell can produce three 10 μm-diameter PMVs in a time window of 2 hours. This amounts to ˜300 μm² membrane area released from the PM of a single cell. Thus, a culture flask holding ˜1×10⁶ adherent cells will yield 3 million PMVs with a mean diameter of 10 μm, corresponding to 314 mm² membrane area.

We also estimated the rate of membrane release by microscopically observing the growth time of one cell-attached PMV at room temperature. Assuming the production of 3 PMVs, an expansion by 5 μm in diameter (5 μm→10 μm) in 30 min corresponds to a membrane release rate of ˜8 μm²/min per cell. For comparison, endocytosis rates are in the range of ˜5 μm²/min per cell. It can be speculated that a cell can release even more PMVs over a longer incubation period, since after removal of the first PMV generation (˜12 hours) an additional incubation round of 12-24 hours, using fresh vesiculation solution, still yields a large amount of PMVs. However, the third generation of PMVs, which was harvested 60 hours after the first incubation round, has a considerably smaller mean diameter, indicating the depletion of available membrane stores.

After the completion of this step a crude polydisperse PMV fraction is obtained with sizes of PMVs ranging up to about ten micrometers in diameter (FIG. 1F). Such PMVs can find great use in several structural and functional assays. Specifically in assays were cell-sized objects are used including high-throughput and high-content analysis.

For applications requiring ultrapure PMV fractions, the crude PMV-containing solution has to be purified, as it contains a range of substances which might contaminate e.g. proteomic analysis. First, PMVs have to be separated from other membranous particles, like detached cells, and cell debris. Since PMVs are filled with cytosol and have a similar size as cells, a large fraction will pellet together with cellular material during centrifugation, hindering effective separation. To avoid this, we utilize the difference in density of PMVs compared to cells. For that, the PMV solution (FIG. 2A) is transferred into a centrifuge tube and underlaid with a high density sucrose phase (FIG. 2B). This is done by carefully adding 2M sucrose underneath the PMV solution. Then, centrifugation is performed for 15 min in a swing-out rotor at 500×g. PMVs accumulate at, but do not cross the buffer/sucrose phase boundary whereas cells pellet at the bottom of the tube, ensuing an almost complete separation of cells and PMVs (FIG. 2C).

Cells can also be removed from the PMV solution by filtering methods, e.g. filters with pore sizes of several micrometers.

Further contaminating material are soluble proteins that likely are released from cells during the vesiculation procedure and from collapsed PMVs. Also, the vesiculation agent FA might hamper efficiency of the downstream protease digestion due to its protein crosslinking activity. It may furthermore for the same reason complicate both functional as well as structural assays. Accordingly, the sample was aspirated (FIG. 2D) and transferred to a dialysis typing (FIG. 2E) and dialyzed using a high cutoff dialysis tube (1 MDa). Dialysis was performed for 8-12 hours against buffer containing 10 mM HEPES and 140 mM NaCl. After the completion of the dialysis the vesicles were transferred to an Eppendorf vial (FIG. 2F)

The method as described above yields a fairly polydisperse PMV fraction with sizes of PMVs ranging up to about ten micrometers in diameter that can be utilized in several structural and functional assays, notably proteomic assays to screen for protein expression, target identification, and many more as further detailed below.

The ultrapure PMV fraction can then be further processed in a number of ways depending on application. Thus, the chemical makeup of PMVs such as modification of sugar residues, membrane proteins, and the membrane itself etc, can be tailored for each case. Colloid size can be an important parameter, and often monodisperse fractions with certain chemical modifications of the membrane proteome are desired. In the following, we describe on-chip, and in-solution processing steps of the ultrapure PMV fraction with the purpose of performing proteomic assays. The processing consists of five steps where several steps are optional depending on the particular application area.

1. Alkylation and reduction of membrane proteins

2. Alkaline wash to disrupt non-covalent protein-protein interactions.

3. Ultrasonication to release intravesicular contaminants and form small vesicles

4. Ultracentrifugation to clean PMV fraction

5. Rinsing and dispersion in ammonium bicarbonate buffer

These steps are referred to by the Arabic numerals in the following text:

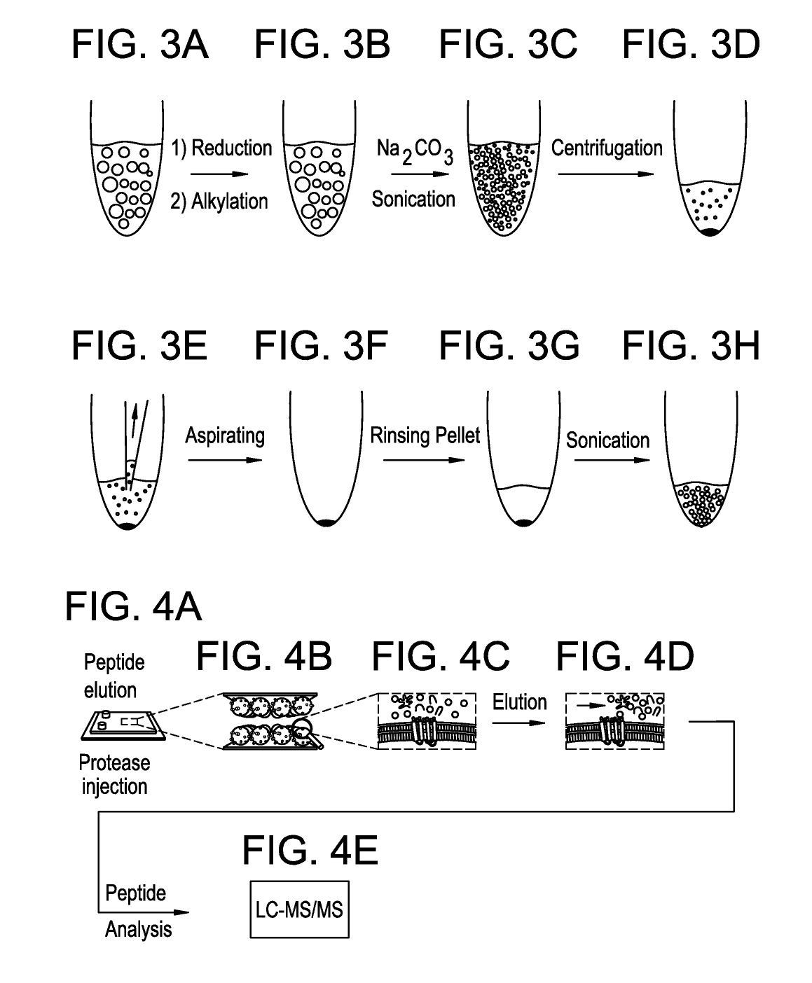

1. Alkylation and reduction of membrane proteins. Surface-exposed membrane proteins in a dialyzed sample of polydisperse PMVs (FIG. 3A) are reduced with 10 mM DTT and alkylated with 50 mM iodoacetamide to break disulfide bonds, with the purpose of making more cleavage sites available for digestion and to prevent protein aggregation (FIG. 3B).

2. Alkaline wash to disrupt non-covalent protein-protein interactions. Second, a high-pH washing step (pH 11, Na₂CO₃) disrupts noncovalent protein-protein interactions, dissociating cytosolic proteins from the membrane (FIG. 3C).

3. Step 2 is performed in combination with ultrasonication to release intravesicular contaminants and form small vesicle. This step also includes extensive sonication which causes PMVs to disrupt and reseal as smaller vesicles, consequently releasing the cytosolic interior into the PMV solution.

4. Ultracentrifugation to clean the PMV fraction. In order to remove this additional contamination source, the PMV membranes are pelleted by ultracentrifugation at 100,000×g, and the supernatant is removed (FIG. 3D).

5. Rinsing and dispersion in ammonium bicarbonate buffer (FIGS. 3E-G) Finally, the membrane pellet is rinsed and dispersed by sonication in 20 mM ammonium bicarbonate buffer and is ready for digestion (FIG. 3H).

After processing of the PMV fraction according to steps 1-5 as described above, we have now obtained an ultra-pure monodisperse fraction of small-sized PMVs. This ultra-pure fraction of monodisperse PMVs can be used for a great number of applications including structural and functional assays.

In the following we describe how this fraction is employed for proteolytic digestion by enzymes with the aim of performing membrane proteomic analysis by LC-MS/MS. We use two different digestion protocols. One is performed in solution and the other is performed using immobilized PMVs in a flowcell as further detailed below.

1. In-solution digestion of membrane proteins in PMVs

2. Digestion of membrane proteins on immobilized PMVs

Digestion may be performed in-solution, as well as after PMV-immobilization in a flowcell. For in-solution digestion, trypsin is added to the processed PMV solution, and the peptides are separated from the membranes by low cut-off filtering.

The working principle of the flowcell is based on solid-phase immobilization of PMVs allowing for simple buffer/reagent exchange, and sample handling (FIGS. 4A-D). The PMV solution is injected into the flowcell, where membranes, but also proteins adhere to the surface. Injection of the trypsin solution initiates digestion of protein domains which are exposed on the surface of immobilized PMVs (FIGS. 4A-D). As some soluble protein contaminants are immobilized and many are washed out during repeated washing cycles without sacrificing the membrane protein fraction the flowcell also provides a purification step providing clean peptide fractions. Finally, the peptides are eluted and analyzed by LC-MS/MS.

### Example 2

### Isolation and Purification of Plasma Membrane Vesicles from NG-108 Cells and Subsequent LC-MS/MS Proteomic Analysis

Isolation and purification of plasma membrane vesicles. NG108-15 cells were grown to confluence using DMEM (4,5 g/L glucose, 2mM L-glutamine) with 10% FCS. Vesiculation was performed as previously described^{20,29} with some modifications. Briefly, the confluent cell layer was washed twice with 10 mM HEPES, 140 mM NaCl, pH 7.4. Cells were incubated with 2-4 mL vesiculation buffer (10 mM Hepes, 140 mM NaCl containing 2 mM CaCl₂, 2 mM DTT, and 25 mM formaldehyde, pH 7.4), where the incubation time was chosen between 8-16 hours at 37° C., with gentle shaking (60-80 cycles/minute). The supernatant was collected from the flasks and pooled in a 15 mL conical tube. In order to remove cells that have detached from the surface, the solution was underlaid with 2 mL 2M sucrose and centrifuged 15 min at 4° C. with 500×g using a swing-out rotor. The supernatant was collected, and it was noted that the majority of large blebs were found in the solution close to the sucrose phase. Next, PMVs were dialyzed, where the Spectrapor Biotech 1000 kDa MWCO CE membrane (Spectrum Labs, Breda, NL) proved to be the most effective dialysis material to remove residual vesiculation buffer components, and low molecular weight proteins prior to analysis. It was noted that the recovery of PMVs was poor after dialysis using a RC (regenerated cellulose) membrane, but PMV yield was stable using a CE (cellulose ester) membrane. We speculate that PMVs adhere to the RC membranes, considerably decreasing the yield. Dialysis was usually performed for 8-12 h at 4 C against 2 L of 10 mM Hepes and 140 mM NaCl, pH 7.4.

Downstream Processing and Proteolytic Digestion of PMVs

The purified PMV solution was processed for optimization of downstream analysis. Reduction was performed with DTT (10 mM final conc, 56° C., 1 hr). Subsequent alkylation was performed with iodoacetamide (50 mM final cone, RT, 1 hr). To the PMV solution Na₂CO₃ was added to a final concentration of 100 mM (pH 11), followed by bath-sonication on ice for ˜30 minutes. Next, membranes were collected by centrifugation (100,000×g, 60 mins). After removal of the supernatant and careful rinsing, the pellet was resuspended and dispersed by sonication in 20 mM ammonium bicarbonate, pH 8. We then used either of two different methods to analyse the protein content: 1) The PMV suspension was digested in-solution using trypsin (0.005 mg/mL, 37° C., 16 hours), followed by filtering (Anotop, 20 nm filter). The filtered peptide solution was analyzed by LC-MS/MS, as described below; 2) The PMV suspension was processed and injected into a LPI™ FlowCell (Nanoxis A B, Göteborg, Sweden) where the vesicles were immobilized Immobilized membrane vesicles were washed by rinsing the flow cell with 300 mM NaCl, 10 mM Tris, pH 8 and then 20 mM ammonium bicarbonate, pH 8. The membrane proteins in the immobilized vesicles were digested by incubating the sample with trypsin (0.005 mg/mL in 20 mM ammonium bicarbonate, pH 8) for 2 h at 37° C. The resulting peptide solution was eluted with 20 mM ammonium bicarbonate, pH 8, and analyzed by LC-MS/MS.

LC-MS/MS and Bioinformatics.

Peptides were analyzed by LC-MS/MS at the Proteomics Core Facility at Göteborg University. Prior to analysis, the sample was vacuum centrifuged to dryness and reconstituted in 20 μL 0.1% formic acid in water. The sample was centrifuged at 13,000×g for 15 min and 17 μL was finally transferred to the autosampler of the LC-MS/MS system. For the liquid chromatography, an Agilent 1100 binary pump was used and the tryptic peptides were separated on a 200·0.05 mm i.d. fused silica column packed in-house with 3 μm ReproSil-Pur C18-AQ particles (Dr. Maisch, GmbH, Ammerbuch, Germany). Sample (2 μL) was injected and the peptides were first trapped on a precolumn (45˜0.1 mm i.d.) packed with 3 μm C18-bonded particles. A 40 min gradient consisting of 10-50% acetonitrile in 0.2% formic acid was used for separation of the peptides and the flow through the column was reduced by a split to approximately 100 nL/min. Mass analyses were performed in a 7-T LTQ-FT mass spectrometer (Hybrid Linear Trap Quadrupole—Fourier Transform) (Thermo Electron) equipped with a nanospray source modified in-house. The instrument was operated in the data-dependent mode to automatically switch between MS and MS/MS acquisition. MS spectra were acquired in the FT-ICR while MS/MS spectra were acquired in the LTQ-trap. For each scan of FT-ICR, the six most intense, doubly or triply protonated ions were sequentially fragmented in the linear trap by collision induced dissociation (CID). Already fragmented target ions were excluded for MS/MS analysis for 6 s. All tandem mass spectra were searched by MASCOT (Matrix Science) against the rodent subset of the SwissProt database. The search parameters that were used were: 5 ppm mass tolerance for precursor ion masses and 0.5 Da for product ion masses; digestion with trypsin; a maximum of one missed tryptic cleavage; variable modifications included oxidation of methionine and carbamidomethylation of cysteines. Only peptides with Mascot expectation value less than 0.05 were considered. Criteria for protein identification included detection of at least 2 unique identified peptides, but single peptide identifications were allowed if the peptide was reproducibly detected. Peptides shared between protein identifications were not included. Subcellular location was assigned based on information obtained from the UniProt database, aided by information gathered from ChromatinDB (http://www.chromdb.org/), subcellular location prediction programs Cello (http://cello.life.nctu.edu.tw/) and ProteomeAnalyst (http://pa.cs.ualberta.ca:8080/pa/), and literature providing proteomic and subcellular analysis data of proteins by LC-MS/MS. Proteins anchored to the bilayer through a transmembrane domain or lipid modification were identified based on Uniprot annotation.

Microsomal Membrane Preparation.

NG108-15 cells were washed with PBS, briefly swollen in 1 mM NaHCO₃ and mechanically disrupted in a tight-fitting Dounce homogenizer with 20 strokes. Nuclei and cell debris were removed by centrifugation (400×g, 5 mins). The supernatant, containing the microsomal membrane fraction, was supplemented with Na2CO₃ to a final concentration of 100 mM (pH 11). Membranes were pelleted by centrifugation (100,000×g, 60 mins). After removal of the supernatant, the membrane pellet was resuspended and dispersed in 300 mM NaCl, 10 mM Tris, pH 8, using a tip sonicator (VibraCell Model 501, Sonics & Materials Inc., USA). The membrane vesicle sample was injected into the LPI™ FlowCell, following the same procedure used for analysis of the microsomal membrane preparation.

Identified Membrane Proteins in PMVs

To determine the sub cellular origin of the PMV membrane, we investigated the sub cellular location of the membrane proteins found therein. Five independent PMV samples have been analyzed, resulting in a total of 274 protein identifications. According to the sources we use to annotate membrane association and sub cellular location 43 PMV proteins are anchored to the membrane by at least one a-helical domain or a lipid anchor (Table 1), and 44 are associated with the membrane by other interactions. 40 of the anchored membrane proteins are found to be located to the PM (90%), of which 32 proteins (74%) are unique to the PM. For the remaining 191 proteins we could not identify any membrane association, and presume that these are soluble proteins originating from inside PMVs.

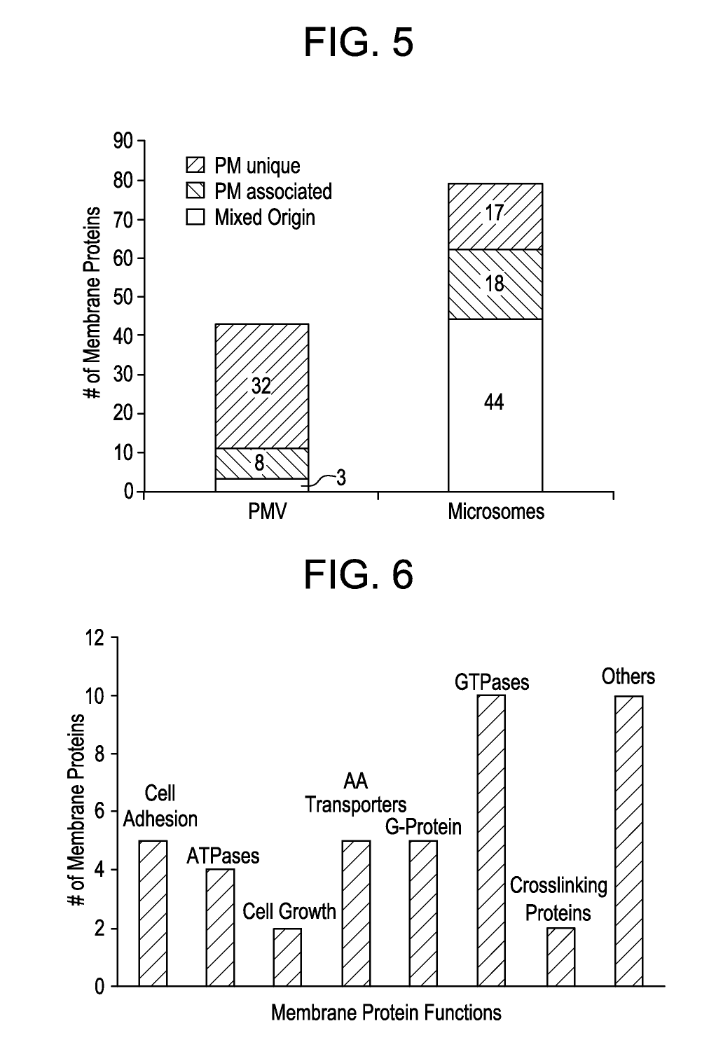

For comparison, we performed also a microsomal preparation of the NG108-15 cell line. This is a standard method to isolate cellular membranes by cell lysis and removal of nuclei and soluble proteins. Two microsomal preparations were analyzed, resulting in a total of 308 protein identifications. 79 proteins are anchored to the membrane by at least one a-helical domain or a lipid anchor, and 57 are associated with the membrane by other interactions. 35 of the anchored membrane proteins are found to be located to the PM (44%), of which only 17 proteins (20%) are unique to the PM (FIG. 5). Compared with the microsomal preparation, the PM protein content of the PMV membrane fraction is much higher (90%).

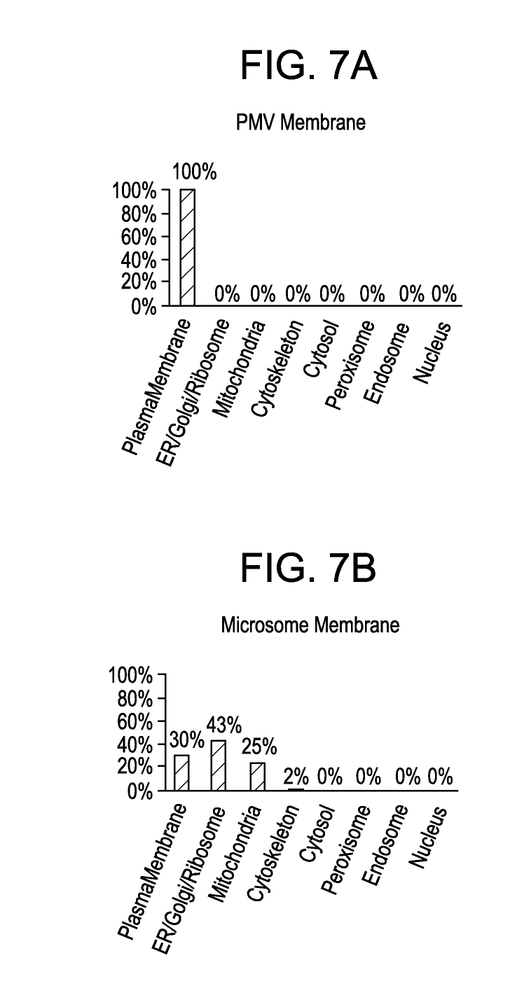

Among the identified membrane proteins in PMVs, GTPases and G-Proteins are predominantly found. Amino acid transporters, ion transporters, as well as proteins responsible for cell adhesion, and growth are also represented (FIG. 6). Notably, putative plasma membrane-cytoskeletal crosslinking proteins were also identified, indicating that the vesiculation process might cause disassociation of these proteins from the cytoskeleton. More details regarding the comparative sub cellular distribution of identified membrane proteins are found in FIG. 7, also comparing microsomal and PMV membrane fractions. In our PMV analyses, we could also identify 191 soluble proteins, many of them ribosomal and cytosolic, which originate from the PMV interior. Presumably, these are released during the processing steps after dialysis of the PMV sample. The first sonication step causes the micron-sized PMVs to disrupt and reseal, releasing the cytosolic interior. Ribosomes seem to be abundant in the PMV solution and due to their large size, they can not be removed by dialysis, and are likely to be pelleted together with the processed PMV membranes in the ultracentrifugation step. Also, as the membrane pellet is undergoing an additional sonication step just prior to digestion, additional release of cytosolic proteins might occur, which could add to the soluble protein count in the obtained result. Further optimization, like tuning of centrifugation steps to remove ribosomal proteins, or finding alternatives to the last sonication step might bring these contamination sources down to a minimum.

### Example 3

### Extraction of Lipid Components

CHO-K1 cells were cultured to 95% confluency in T175 flasks. Media was removed from the flasks, and the cells were washed several times with 150 mM NaCl, 10 mM HEPES, 2 mM CaCl₂, pH 7.4. To induce plasma membrane vesiculation, 6 mL of a solution of 25 mM formaldehyde, 2 mM DTT, 150 mM NaCl, 10 mM HEPES, 2 mM CaCl₂, pH 7.4, was added to each flask. Vesiculation was done for 2 hours at 37 C, with gentle rocking of the cell flasks. After vesiculation, the solutions containing plasma membrane vesicles were collected from the flasks and pooled. The solution was passed through a 40 μm pore filter to remove aggregates of cells, and through a 5 μm filter to remove single cells. The solution was then frozen at −20C. The blebb solutions from different cell batches were pooled into larger batches for extraction. The total blebb solution volume was measured and used to calculate the organic solvent volumes for extraction. The first step was to add NH₄Ac (ammonium acetate) to a final concentration of 10 mM. A modified Bligh-Dyer extraction protocol was used where the ratios of solvents were set to 2:1:0.8 (MeOH:DCM:NH₄Ac (10 mM)). Methanol (MeOH) and dichloromethane (DCM) was then added to the blebbsolution. No phase separation was seen and the solution was tipsonicated using a Vibra Cell (model 501) from Sonics & Materials Inc equipped with a 13 mm probe tip. Sonication was performed during 2 minutes at 30% amplitude setting with 7 second pulses and 5 seconds rest in between to reduce heating of the sample. Phase separation was induced by adding 40 ml DCM and 10 ml NH₄AC (120 mM) and the DCM phase was collected. Again, 10 ml of NH₄Ac (120 mM) was added and the DCM phase was collected. 50 ml of DCM was then added to the MeOH/NH₄Ac phase and was tipsonicated as above. After tipsonication, 10 ml of NH₄Ac (120 mM) and 50 ml DCM was added to the solution. After shaking and phase separation the DCM phase was collected. 50 ml DCM, 50 ml MeOH and 10 ml NH₄Ac (120 mM) was added to the remaining MeOH/NH₄Ac phase. After shaking, the DCM phase was collected. Finally, 10 ml NH₄Ac (120 mM) was added to the remaining MeOH/NH₄Ac phase and the DCM was collected. After storage of the pooled DCM phase in −20 degrees over night, a MeOH/NH₄Ac phase could be separated from the DCM phase prior rotaevaporation of the DCM phase. The DCM phase was rotaevaporated and the dried residue was weighed. It was estimated that blebs produced from roughly 80 million cells gave 1 mg of dry lipid after extraction. Furthermore, the dried lipid residue was checked for protein contaminants using SDS-PAGE. The lipid residue from blebs emanating from roughly 10 million was first reconstituted in 2×SDS-PAGE sample buffer (4% SDS). The sample was gently tipsonicated using a Vibra Cell (model 501) from Sonics & Materials Inc equipped with a 2 mm tip (30 second sonication time, 2 second pulses and 2 second rest time in between at 5% amplitude setting). The sample was then heated in waterbath (≦100 degrees Celcius) followed by swirling for 5 minutes. Resuspended lipid was then diluted with MQ before running the sample on a standard 10% acrylamide gel for 1 hour. The result indicates that the lipid extract is free from protein contaminants.

Discussion

Our method exploits the ability of cells to shed the PM from its surface in the form of micron-sized vesicles. Its principal advantages are the high purity of the membrane preparation with regard to PM content, as well as easy handling procedures. PMVs do not contain organelles or cytoskeletal structures, but are filled with cytosolic components. Due to the fact that PMVs originate solely from the PM, they provide an excellent platform for an extensive range of applications, especially in proteome science. One can control the membrane and interior protein composition of PMVs by applying molecular biology techniques on the cell culture beforehand, such as transfection, recombinant or overexpression of proteins, fluorescent labelling, gene silencing etc. This would be beneficial for comparative proteomic studies of the PM and give insight on its dynamical behaviour. For example, the spatiotemporal behaviour of PM proteins and the dynamical exchange of membranes with the endomembrane system are important issues that could be addressed. Our presented method to analyze the PM proteome could help answering the question if internal membrane stores are recruited for PMV formation after prolonged incubation time (>24hours), by analyzing and comparing the different PMV generations as, for example, a comparatively higher fraction of ER/Golgi proteins may be found in later PMV generations. The purification protocol may be applied to a wide variety of cell lines^{21,22}, as other mammalian cell lines, such as HEK293 and CHO-K1, are also able to produce PMVs in large numbers when exposed to the vesiculation solution (data not shown). This renders the method a promising technique to analyze and compare PM proteomes of different cell lines, as well as comparing varying protein expression profiles of a specific cell line of interest.

We envision our presented technique as a powerful tool for proteomic analyses of mammalian PMs as it is a way to obtain PMs of very high purity with regard to membrane protein content, and, when combined with molecular biology techniques, provides a powerful means to study the dynamical nature of the plasma membrane proteome. In addition, as membrane and cytosolic components are integrative in each single PMV, they constitute a versatile simplistic cell model, enabling studies of more complex cellular processes. For example, a proteomic analysis of the PMV interior can be extremely useful for e.g. investigation of membrane protein activities coupled with cytosolic proteins.

### INCORPORATION BY REFERENCE

The contents of all references, patents, pending patent applications and published patents, cited throughout this application are hereby expressly incorporated by reference.

### EQUIVALENTS

Those skilled in the art will recognize, or be able to ascertain using no more than routine experimentation, many equivalents to the specific embodiments of the invention described herein. Such equivalents are intended to be encompassed by the following claims.

TABLE 1

| SP Acc# | Gene | Description | #Peps | Mem | Location |
|---|---|---|---|---|---|
| O35566 | Cd151 | CD151 antigen | 1 | TM, LA | PM |
| O35874 | Slc1a4 | Neutral amino acid transporter A | 3 | TM | PM |
| O88507 | Cntfr | Ciliary neurotrophic factor receptor alpha precursor | 1 | LA | PM |
| P06837 | Gap43 | Neuromodulin | 8 | LA | PM |
| P09055 | Itgb1 | Integrin beta-1 precursor | 3 | TM | PM |
| P09242 | AlpI | Alkaline phosphatase, tissue-nonspecific isozyme precursor | 3 | LA | PM |
| P10852 | Slc3a2 | 4F2 cell-surface antigen heavy chain | 21 | TM | PM |
| P11505 | Atp2b1 | Plasma membrane calcium-transporting ATPase 1 | 8 | TM | PM |
| P11627 | L1cam | Neural cell adhesion molecule L1 precursor | 2 | TM | PM |
| P13596 | Ncam1 | Neural cell adhesion molecule 1, 140 kDa isoform precursor | 7 | TM | PM, CSk |
| P14094 | Atp1b1 | Sodium/potassium-transporting ATPase subunit beta-1 | 2 | TM | PM |
| P18572 | Bsg | Basigin precursor | 5 | TM | PM |
| P21279 | Gnaq | Guanine nucleotide-binding protein G(q) subunit alpha | 4 | LA | PM |
| P21995 | Emb | Embigin precursor | 4 | TM | PM |
| P26645 | Marcks | Myristoylated alanine-rich C-kinase substrate | 4 | LA | PM, CSk, CSol |
| P27601 | Gna13 | Guanine nucleotide-binding protein alpha-13 subunit | 2 | LA | PM |
| P28656 | Nap1l1 | Nucleosome assembly protein 1-like 1 | 4 | LA | N, CSol |
| P28667 | Marcksl1 | MARCKS-related protein | 2 | LA | PM |
| P32037 | Slc2a3 | Solute carrier family 2, facilitated glucose transporter member 3 | 3 | TM | PM |
| P35279 | Rab6a | Ras-related protein Rab-6A | 1 | LA | ER, G, ES |
| P35762 | Cd81 | CD81 antigen | 1 | TM | PM |
| P38402 | GNAl2 | Guanine nucleotide-binding protein G(i), alpha-2 subunit | 3 | LA | PM |
| P40240 | Cd9 | CD9 antigen | 2 | TM, LA | PM |
| P51150 | Rab7a | Ras-related protein Rab-7a | 3 | LA | ER, G, ES |
| P51912 | Slc1a5 | Neutral amino acid transporter B(0) | 7 | TM | PM |
| P53986 | Slc16a1 | Monocarboxylate transporter 1 | 6 | TM | PM |
| P60766 | Cdc42 | Cell division control protein 42 homolog precursor | 4 | LA | PM, N |
| P61027 | Rab10 | Ras-related protein Rab-10 | 1 | LA | PM, ER, G |
| P62492 | Rab11a | Ras-related protein Rab-11A | 3 | LA | PM, ES |
| P62821 | Rab1A | Ras-related protein Rab-1A | 8 | LA | PM, ER, G |
| P62835 | Rap1a | Ras-related protein Rap-1A precursor | 1 | LA | PM |
| P84078 | Arf1 | ADP-ribosylation factor 1 | 2 | LA | PM, ER, G |
| P97370 | Atp1b3 | Sodium/potassium-transporting ATPase subunit beta-3 | 1 | TM | PM |
| Q06806 | Tie1 | Tyrosine-protein kinase receptor Tie-1 precursor | 1 | TM | PM |
| Q61735 | Cd47 | Leukocyte surface antigen CD47 precursor | 1 | TM | PM |
| Q80SZ7 | Gng5 | Guanine nucleotide-binding protein G(I)/G(S)/G(O) subunit | 1 | LA | PM |
| | | gamma-5 precursor | | | |
| Q8R4A8 | GNAS | Guanine nucleotide-binding protein G(s) subunit alpha | 7 | LA | PM |
| Q8VDN2 | Atp1a1 | Sodium/potassium-transporting ATPase subunit alpha-1 precursor | 24 | TM | PM |
| Q91XV3 | Basp1 | Brain acid soluble protein 1 | 8 | LA | PM |
| Q99JI6 | Rap1b | Ras-related protein Rap-1b precursor | 1 | LA | PM |
| Q9QUI0 | Rhoa | Transforming protein RhoA precursor | 4 | LA | PM, CSk |
| Q9R1Q7 | Plp2 | Proteolipid protein 2 | 1 | TM, LA | PM |
| Q9Z127 | Slc7a5 | Large neutral amino acids transporter small subunit 1 | 7 | TM | PM |

1. A method for producing plasma membrane vesicles comprising:
contacting a cell with a vesiculation agent;
thereby producing plasma membrane vesicles,

2. The method of claim 1, further comprising mechanically agitating the cells,

3. The method of claim 1, wherein the cells are adherent cells.

4. The method of claim 1, wherein the cells are in suspension.

5. The method of claim 1, wherein the cells are mammalian cells,

6. The method of claim 1, wherein the vesiculation agent comprises a sulfhydryl blocking agent.

7. The method of claim 6, wherein the sulfhydryl blocking agent is selected from the group consisting formaldehyde, pyruvic aldehyde, acetaldehyde, glyoxal, glutaraldehyde, acrolein, methacrolein, pyridoxal, N-ethyl malemide (NEM), malemide, chloromercuribenzoate, iodoacetate, potassium arsenite, sodium selenite, thimerosal (merthiolate), benzoyl peroxide, cadmium chloride, hydrogen peroxide, iodosobenzoic add, meralluride sodium, (mercuhydrin), mercuric chloride, mercurous chloride, chlormerodrin (neohydrin), phenylhydrazine, potassium tellurite, sodium malonate, p-arsenosobenzoic add, 5,5′-diamino-2,2′-dimethyl arsenobenzene, N,N′-dimethylene sultonate disodium salt, iodoacetamide, oxophenarsine (mapharsen), auric chloride, p-chloromercuribenzoic acid, p-chloromercuriphenyisullonic acid, cupric chloride, iodine merbromin (mercuro chrome)porphyrindine, potassium permanganate, mersalyl (salyrgan), silver nitrate, strong silver protein (protargol), and uranyl acetate.

8. The method of claim 1, wherein the vesiculation agent comprises dithiothreitol (DTT) and formaldehyde.

9. The method of claim 1, wherein the vesiculation agent is a cell toxin.

10. The method of claim 9, wherein the cell toxin is cytochalasin B or melittin

11. The method of claim 1, wherein the cells are mechanically agitated by a shaker.

12. The method of claim 1, wherein the cells are mechanically agitated by ultrasonication.

13. The method of claim 1, further comprising washing cells to remove culture medium prior to contacting cells with the vesiculation agent.

14. The method of claim 1, further comprising purifying the plasma membrane vesicles.

15. (canceled)

16. A method for making high purity plasma membrane vesicles comprising one or more of the following steps: contacting the plasma membrane vesicles of claim 1 with an alkylating and reducing agent; contacting the plasma membrane vesicles of claim 1 with an alkaline solution: using ultrasonication on the plasma membrane vesicles of claim 1 to release intravesicular contaminants; using ultracentrifugation on the plasma membrane vesicles of any one of claim 1 to clean the plasma membrane vesicles; and washing the plasma membrane vesicles of claim 1 with a buffer solution.

17. The method of claim 16, wherein the alkylation reagent is iodoacetamide.

18. The method of claim 16, wherein the alkaline solution is at least pH 11.

19. The method of claim 16, wherein the alkaline solution is Na₂CO₃ or NaOH.

20. (canceled)

21. (canceled)

22. The method of any one of claim 1-21, wherein the plasma membrane vesicles comprise transmembrane proteins.

23. (canceled)

24. The method of claim 22, wherein the transmembrane proteins are selected from the group consisting of enzymes, transporters, receptors, channels, cell adhesion proteins, G proteins, GTPases

25. The method of of claim 1, wherein the plasma membrane vesicles comprise lipid anchored proteins,

26. A method for analyzing the membrane proteome of a cell comprising: contacting the plasma membrane vesicle of claim 1 with one or several proteases; or several proteases in series analyzing the peptides generated by the protease; thereby analyzing the membrane proteome of a cell.

27. (canceled)

28. (canceled)

29. (canceled)

30. (canceled)

31. A method of identifying a modulator of a transmembrane protein comprising: transforming a cell with a nucleic acid molecule encoding a protein of interest;
producing plasma membrane vesicles by the method of claim 1;
contacting the plasma membrane vesicles with a candidate modulator; determining if the candidate modulator is capable of modulating the transmembrane protein; thereby identifying a modulator of a transmembrane protein.

32. (canceled)

33. A method of determining the effect of a compound on the transmembrane proteome comprising: contacting a cell with a compound; producing plasma membrane vesicles by the method of claim 1; analyzing the polypeptides present in the plasma membrane vesicles: thereby determining the effect of a compound on the transmembrane proteome.

34. (canceled)

35. (canceled)

36. (canceled)

37. A method of analyzing the proteins in a plasma membrane vesicles of claim 1, comprising: affixing the plasma membrane vesicles to a surface: contacting the plasma membrane vesicles with one or more proteases;
analyzing the peptides generated to determine the identity of the proteins.

38. (canceled)

39. (canceled)

40. A method for extracting the lipids from a cell membrane comprising:
inducing vesculation of one or more cells; isolating the membrane vesicles; extracting the membrane vesicles with an organic solvent; isolating the lipids from the organic solvent;
thereby extracting the lipids from the cell membrane.

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)
